(19) United States
(12) Patent Application Publication
(10) Pub. No.: US 2024/0216278 A1
(43) Pub. Date: Jul. 4, 2024

(54) METHODS FOR PRODUCING NANOPARTICLE DISPERSIONS

(71) Applicant: leon-nanodrugs GmbH, Munich (DE)

(72) Inventors: Christian DUBIELLA; Julian VOGLER; Pascale CLEMENT; Frank STIENEKER

(21) Appl. No.: 18/558,368

(22) PCT Filed: May 5, 2022
(86) PCT No.: PCT/EP2022/062222

§ 371 Date: Nov. 1, 2023

(30) Foreign Application Priority Data
May 5, 2021 (EP) 21172324.2

(51) Int. Cl. A61K 9/127

(52) U.S. Cl. CPC A61K 9/1271; A61K 9/1277

(57) ABSTRACT

The disclosure presented herein provides methods for producing a dispersion of nanoparticles, as for example liposomes, by a method comprising frontally colliding an organic stream with an aqueous stream at raised pressure.

### FIELD OF THE DISCLOSURE

The disclosure presented herein provides methods for producing a dispersion of lipid carriers, as for example liposomes or lipid nanoparticles, by a method comprising frontally colliding an organic stream with an aqueous stream at raised pressure.

### BACKGROUND

The pharmaceutical and biotech industries have an increasing and urgent need for technologies that can safely and successfully deliver active pharmaceutical ingredients, in particular nucleic acids, for a variety of disease applications including vaccination and gene therapy. Currently, this need is not fully met, as seen by the capacity constraints on SARS-CoV-2 vaccine supply and some concerns on adenovirus technology application in humans.

Lipid carriers such as lipid nanoparticles (LNPs) and associated technology provide a proven, efficient, and safe method of delivering active pharmaceutical ingredients. One of the major issues for LNPs is their manufacture in sufficient quantity and the batch reproducibility standards required for commercialization. Multiple strategies exist for delivery of active pharmaceutical ingredients in LNPs, among them, jet impinging technology is being successfully applied for the production of nucleic acid-based vaccines, but its efficiency and utility is far from optimised.

The terms “liposome” and “lipid nanoparticle” (LNP) are sometimes used interchangeably; however, the former generally refers to a spherical vesicle composed of phospholipids and containing at least one lipid bilayer whereas the structure of an LNP may be less ordered. LNPs have been described as “a new generation of liposomes with a more complex internal lipid architecture with low or minimal internal aqueous presence that is well suited to stable and efficient encapsulation of various genetic payloads”. Additionally, there are other lipid carrier or lipid nanoparticle structures, like e.g. lipoplexes, lipopolyplexes, multilamellar liposomes or solid lipid nanoparticles.

Liposomes are defined as nanosized vesicular structures with particle sizes ranging from 30 nm to several micrometres consisting of an aqueous core surrounded with at least on phospholipid bilayers. Due to their size and hydrophobic and hydrophilic character (besides biocompatibility), liposomes are promising systems for drug delivery. In liposomes, water-soluble drugs can be encapsulated in the aqueous core while the lipid bilayer is responsible for entrapping water-insoluble drugs. Liposome properties differ considerably with lipid composition, surface charge, size, and the method of preparation. Furthermore, the choice of bilayer components determines the ‘rigidity’ or ‘fluidity’ and the charge of the bilayer.

It is known that oral bioavailability of poorly soluble active pharmaceutical ingredients can be increased by reducing the particle size of the active pharmaceutical ingredients. Moreover, application of nanotechnology for nanosizing and/or encapsulation of poorly soluble active pharmaceuticals as well as encapsulation of water-soluble active pharmaceuticals for e.g. parenteral administration showed advantages in terms of e.g. increase in bioavailability, reduction of side effects, passive targeting to tumor sites by enhanced permeation though leaky junctions, etc.

When designing pharmaceutical products, it is advantageous to be able to set the particle size freely, since passive drug targeting becomes possible. Furthermore, pharmaceutical products, in particular when administered parenterally, must be sterile. Thus, for parenteral products, a small particle size, which allows sterile filtration, is useful. Other types of sterilisation (e.g. autoclaving) are very likely to destroy liposomes and are therefore not used.

Various technologies are used for the production of lipid carriers systems.

Solvent/nonsolvent precipitation methods have been found useful for the production of nanoparticles, in particular lipid-based nanoparticles. Solvent/nonsolvent precipitation means that a substance is dissolved in a solvent and collides as a liquid jet with a second liquid jet that contains a nonsolvent, whereby the dissolved substance is precipitated. Microjet reactor technology is used for an automated, continuous approach for nanoparticle synthesis by solvent/nonsolvent precipitation methods. It creates a turbulent mixing zone of solvent and nonsolvent and is therefore particularly suitable for the precipitation of particles in the nanometer range.

EP 2395978 B1 describes the solvent/nonsolvent precipitation in the presence of surface-active molecules using a microjet reactor according to EP 1 165 224 B1. Such a microjet reactor has at least two nozzles (pin-holes) located opposite one another, each with an associated pump and feed line for spraying a liquid medium at a common collision point in a reactor chamber enclosed by a reactor housing. Another opening is provided in the reactor housing through which a gas, an evaporating liquid, a cooling liquid, or a cooling gas can be passed to maintain the gas atmosphere in the reactor chamber or for cooling. A further opening is provided for removing the resulting products and excess gas from the reactor chamber. Thus, a gas, an evaporating liquid or a cooling gas is introduced into the reactor chamber through an opening in order to maintain a gas atmosphere inside the reactor or to cool the resulting products, and the resulting products and excess gas are removed from the reactor chamber through this opening by overpressure on the gas inlet side or by underpressure on the product and gas outlet side. If a solvent/nonsolvent precipitation is carried out in such a microjet reactor a dispersion of precipitated particles is obtained.

In particular for commercial application of the lipid nanoparticles, e.g. for therapeutic approaches or vaccination, it is of high importance to provide a robust method of production that can easily be upscaled and provide a fast production of the desired product. Additionally, it is also of importance that the production process can be adapted to different needs and requirements that may emerge during the process of product development or during upscaling. Since lipid nanoparticles may be suitable carriers, or delivery vehicles for nucleic acids or nucleic acid constructs which can be relevant towards therapeutic applications such as vaccination and gene therapy, the production processes must also be suitable handling these types of active ingredients and molecules.

It is thus an object of the present invention to provide a method for the production of lipid-based nanoparticles that is flexible in terms of adaptation to different kind of products as well as robust, fast and reliably producing the desired product. Another object is to provide a method for producing lipid-based nanoparticles which is versatile for process development. A further object is to provide a method for the production of said particles, based on the use of an impingement reactor which may overcome one or more disadvantages or limitations of jet impingement reactors in the prior art. Further objects of the invention will be clear on the basis of the following description of the invention, examples and claims.

### SUMMARY OF THE DISCLOSURE

In a first aspect, is the invention relates to a method for producing a dispersion of nanoparticles comprising at least one amphiphilic lipid, wherein the method comprises the steps of: a) providing a first stream comprising an organic solvent and at least the amphiphilic lipid; b) providing a second stream comprising an aqueous solvent; c) pumping the first stream under a raised pressure through a first nozzle and pumping the second streams under a raised pressure through a second nozzle into a reaction chamber; wherein the first nozzle is located at an angle of about 180° from the second nozzle; d) colliding the first stream and the second streams frontally in a reaction chamber; and wherein the flow rate ratio between the first stream and the second stream is in a range from about 1:1.5 to below about 1:4.5.

In a further aspect, disclosed herein is a nanoparticle dispersion produced by the method of the first aspect of the invention.

In a further aspect, disclosed herein is a pharmaceutical composition comprising a nanoparticle dispersion produced by the method of the first aspect of the invention.

### DESCRIPTION OF THE DRAWINGS

FIGS. 1A and 1B show the effect of the SPC/Cholesterol ratio on particle size and PDI of liposomes of Example 1, prepared using either a commercial microfluidic mixer (FIG. 1A) or the method of the invention, i.e. jet impinging technology (System B) (FIG. 1B). SPC/Cholesterol weight ratios of 3:1 (33 mol % Cholesterol, ID1, ID3, ID5 and ID7) and 2:1 (50 mol % Cholesterol, ID2, ID4, ID6, ID8) were tested. Data was acquired using dynamic light scattering (DLS) (n=1).

FIG. 2 shows the effect of reactor nozzle size on particle size and PDI of liposomes prepared in Example 1 as observed with a jet impingement technology (System B). The applied nozzle sizes were 100 μm for the first opening (organic stream) and 200 μm for the second opening (aqueous stream) (ID3, ID4); and 200 μm (first opening) and 300 μm (second opening) (ID9, ID10). Data were acquired using dynamic light scattering (n=1).

FIGS. 3A to 3D show the effect of flow rate ratios (FRR) and total flow rate (TFR) on particle size and PDI of liposomes prepared in Example 1. FIG. 3A shows the effect of FRR for a commercial microfluidic mixer, while in FIG. 3B a jet impinging system according to the invention (System B) was used. FIG. 3C shows the effect of TFR for a commercial microfluidic mixer, while in FIG. 3D a jet impinging system according to the invention (System B) was used. Data was acquired using dynamic light scattering (n=1).

FIGS. 4A and 4C depict particle size and PDI characterization of liposomes obtained using a commercial micro fluidic mixer, in reference to https://www.precisionnanosystems.com/docs/default-source/pni-files/app-notes/pni-app-bt-013.pdf?sfvrsn=aa668324_0; Brown A., Thomas A., Shell Ip, Heuck G., Ramsay E., Liposomes, published by Precision NanoSystems Inc. 2018; retrieved 05.05.2021. FIGS. 4B and 4D shows particle size and PDI for liposomes of formulation 2, as described in Example 2 (n=1) prepared using jet impinging method according to the invention (System B). In FIG. 4B, the effect of the flow rate ratio (FRR) is shown (TFR was 90 ml/min). The indicated flow rates on the x-axis of 1.0, 1.5, 2.0, 3.0 and 4.0 refer to flow rates (first/organic stream:second/aqueous stream) of 1:1, 1:1.5, 1:2.0, 1:3.0 and 1:4. FIGS. 4A and 4B shows that increasing flow rate of aqueous second stream against the flow rate of the first organic solvent stream leads to a trend towards decreasing particle sizes and towards increasing PDI. FIG. 4D shows the effect of the TFR (FRR of organic:aqueous streams was 1:2) in liposomes prepared using the jet impingement method, where the particle size remained stable over a TFR ranging from 30 ml/min to 300 ml/min. FIG. 4C shows in comparison that in the microfluidic system, the effect of increasing TFR appears to influence particle sizes.

FIGS. 5A to 5F show the FRR and TFR effects on liposomes prepared according to Example 2 from two different liposomal lipid formulations. TFR was 90 ml/min for the FRR comparison and FRR was 1:2 (organic:aqueous) for the TFR comparison. FIGS. 5A and 5B shows the effect of FRR (FIG. 5A) and TFR (FIG. 5B) on particle size and PDI for liposomes of formulation 1, as described in Example 2 (n=3). FIGS. 5C and 5D shows the effect of FRR (FIG. 5C) and TFR (FIG. 5D) on particle size and PDI for liposomes of formulation 2, as described in Example 2 (n=1). FIGS. 5E and 5F show the 1-week physical stability of the PEGylated liposomes of formulation 1, based on TFR and FRR respectively (left bar: t=0, right bar: t=1 week for particle size diameter (nm); circles: t=0, triangles: t=1 week for PDI). Data was acquired using dynamic light scattering and are displayed as mean±SD (n=3). FIGS. 5G and 5H show the 1-week physical stability of PEGylated liposomes of formulation 2 (n=1) based on TFR and FRR respectively (left bar: t=0, right bar: t=1 week for particle size diameter (nm); circles: t=0, triangles: t=1 week for PDI). Data was acquired using dynamic light scattering.

FIGS. 6A and 6B show Cryo-TEM images of liposome samples obtained at different TFR conditions. FIG. 6A shows hundreds of small unilamellar liposomes mainly stuck to the carbon grid used for sample preparation as well as large bilamellar and multivesicular particles (PDI: 0.17, TFR: 30 ml/min). FIG. 6B shows a mix of small unilamellar and bilamellar liposomes in addition to the predominant particle fraction of small unilamellar vesicles stuck to the carbon grid (PDI: 0.165, TFR: 90 ml/min).

FIG. 7 shows the result of Cryo-TEM analysis of particles of Example 3, obtained by the method of the invention (System B).

FIG. 8 shows the correlation between reactor nozzle size and particle size (Z-average, nm). The reactors are labeled according to the size of the nozzle in diameter (μm). As an example, the reactor 400/200 has a (second) nozzle opening of 400 μm in diameter where the second (aqueous) stream is pumped through, and a (first) nozzle opening of 200 μm diameter, where the first (organic) stream is pumped through.

FIG. 9 shows the correlation between Log pressure (“Log Druck absolut [bar]”; x-axis) and particle size (Z-average [nm], y-axis) at different total flow rates (TFR) (circles: 50 ml/min; stars: 180 ml/min; triangles: 312.5 ml/min). Parameter estimates intercept=80.556; Slope=−3.2365

FIG. 10 shows the correlation between flow rate ratio (FRR) and polydispersity index (PDI; y-axis). Flow rate ratios (FRR) indicated on the x-axis as 1.5, 2, 2.5, 3, 3.5 and 4 refer to flow rates (first/organic stream:second/aqueous stream) of 1:1.5, 1:2, 1:2.5; 1:3, 1:3.5 and 1:4 respectively. The dashed lines show 95% confidence interval (CI) bands.

FIG. 11 depicts gel electrophoresis of Fluc mRNA (APExbio) which was circulated in System B as described in Example 5. Samples were withdrawn after the following timepoints and analyzed in separate lanes, from left to right: L (ladder), t=0; before start, after prime, 2 sec, 5 sec, 10 sec, 20 sec, 30 sec, 1 min, 2 min, 3 min, 4 min, and empty lane. No significant mRNA degradation is observed comparing start t=0 and at 4 mins (corresponding to ca. 120 passages through the equipment).

FIG. 12 shows results of the in vitro transfection assay described in Example 6 using 10 μg mRNA (according to EE % assay) per transfection. All data n=6, non-parametric, Kruskal-Wallis post AVG+/−SD, **p<0.01. High transfection rates were observed for both tested groups FLuc-mRNA loaded LNPs generated by jet impingement and by microfluidic mixing respectively.

FIG. 13 is a graphical depiction of the results of the in vivo transfection assay described in Example 6, and shows the in vivo gene delivery quantification for total body over time (post dosing). As shown in the Figure, FLuc expression was observed to peak at 6 h post-dosing and declined thereafter, similarly in both tested groups.

FIG. 14, which is not to scale, depicts a jet impingement reactor (1) according to one embodiment of the disclosure. The reaction chamber (6) defined by the interior surface (2) of the chamber wall (3) is substantially spherical, except for the two fluid inlets (4) and the fluid outlet (7). The fluid inlets (4) are arranged at opposite positions on a first central axis (x) of the reaction chamber (6) and point at one another. Each of the fluid inlets (4) comprises a nozzle (5), which is a plain orifice nozzle in this embodiment. The fluid outlet (7) is positioned on a second central axis (y) which is perpendicular to the first central axis (x). The distance (d) between the two nozzles (4) is substantially the same as the diameter of the spherical reaction chamber (6).

FIG. 15 depicts a fluid inlet connector (10) according to one embodiment of the disclosure. The connector (10) has an upstream end (11), a downstream end (12) holding a nozzle (13) at a downstream position of the downstream end (12), and a fluid conduit (14) for conducting a fluid from the upstream end (11) to the downstream end (12). The fluid inlet connector (10) is designed to provide a fluid inlet for a jet impingement reactor (not shown) according to the invention, and to be reversibly insertable into the wall such of such reactor. The figure is not to scale.

FIG. 16, which is also not drawn to scale, depicts a fluid inlet connector (20) according to another embodiment of the present disclosure. Also, this connector (20) is designed to be reversibly insertable into the wall of a jet impingement reactor (not shown) according to the invention, such as to provide a fluid inlet. It has an upstream end (21), a downstream end (22) holding a nozzle (23) at a downstream position of the downstream end (22), and a fluid conduit (24) for conducting a fluid from the upstream end (21) to the downstream end (22).

### DETAILED DESCRIPTION OF THE INVENTION

In a first aspect, the invention relates to a method for producing a dispersion of nanoparticles comprising at least one amphiphilic lipid, wherein the method comprises the steps of: a) providing a first stream comprising an organic solvent and the amphiphilic lipid; b) providing a second stream comprising an aqueous solvent; c) pumping the first stream under a raised pressure through a first nozzle and pumping the second streams under a raised pressure through a second nozzle into a reaction chamber; wherein the first nozzle is located at an angle of about 180° from the second nozzle; d) colliding the first stream and the second streams frontally in a reaction chamber; and wherein the flow rate ratio between the first stream and the second stream is in a range from 1:1.5 to about 1:4.5.

The present subject matter may be understood more readily by reference to the following detailed description, which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. The term “plurality”, as used herein, means more than one. When values are expressed as approximations, by use of the antecedent “about,” it is understood that the particular value forms another embodiment. All ranges are inclusive and combinable. In some embodiments, the term “about”, refers to a deviance of between 0.0001-10% from the indicated number or range of numbers. In some embodiments, the term “about”, refers to a deviance of up to 25% from the indicated number or range of numbers. The term “comprises” means encompasses all the elements listed, but may also include additional, unnamed elements, and it may be used interchangeably with the terms “encompasses”, “includes”, or “contains” having all the same qualities and meanings. The term “consisting of” means being composed of the recited elements or steps, and it may be used interchangeably with the terms “composed of” having all the same qualities and meanings.

### Dispersions of Nanoparticles

The term “dispersion”, as used herein, refers to a system in which nanoparticles comprising an amphiphilic lipid are dispersed in a continuous phase of another material, such as an aqueous liquid. In some embodiments, a dispersion comprises a colloidal solution, a colloid, or a suspension.

A skilled artisan would appreciate that a “nanoparticle”, also referred to as an ultrafine particle, is usually defined as a particle of matter smaller than about 1000 nm. In a nanoparticle dispersion, such as those disclosed herein, there is a variance in the nanoparticles' particle size, and therefore it is useful to refer to the nanoparticles “average particle size”, as well as to the “polydispersity index” or “PDI”. The term “average particle size”, when used herein to describe the size of nanoparticles, refers to the z-average diameter. Throughout this document, the z-average diameter is measured by Dynamic Light Scattering. Dynamic light scattering (DLS) is a technique in physics commonly known to be used to determine the size distribution profile of small particles in suspension. For the measurement, Zetasizer devices from Malvern Panalytical Ltd., e.g. Malvern Zetasizer Advance Range or Zetasizer AT or Zetasizer ZS or ZS90, or other suitable devices, like DLS technology from Wyatt Technology Corporation can be used, or devices like Stunner, from Unchained Labs can also be used. For the determination of the average particle size a Malvern Zetasizer Nano ZS or Unchained Labs Stunner may be used at constant temperature of 25° C. during the measurement. The z-average diameter, together with the polydispersity index (PDI), is preferably calculated from the cumulants analysis of the DLS measured intensity autocorrelation function as defined in ISO22412:2008. PDI is a dimensionless estimate of the width of the particle size distribution, scaled from 0 to 1. According to Malvern Instruments, samples with PDI≤0.4 are considered to be monodisperse.

In some embodiments, the nanoparticles of the dispersion have an average particle size in the range from 1 to 500 nm, from 5 to 400 nm, from 10 to 200 nm, or from 20 to 100 nm, or from 30 to 90 nm, or from 40 to 80 nm. In some embodiments, a nanoparticle comprises an average particle size below 100 nm, or below 90 nm, or below 80 nm, or below 70 nm. In some embodiments, a nanoparticle comprises an average particle size in the range of 10 to 20, 20 to 30, 30 to 40, 40 to 50, 50 to 60, 60 to 70, 70 to 80, 80 to 90, or 90 to 100 nm. In some embodiments, a nanoparticle comprises an average particle size of about 10, 20, 30, 40, 50, 60, 70, 80, 90, or 100 nm.

In some embodiments, the nanoparticles of the dispersion have a polydispersity index (PDI) below 0.4, or below 0.3, or below 0.25, or below 0.2, or below 0.1. In some embodiments, the nanoparticles PDI is in the range from 0.01 to 0.05, or from 0.05 to 0.1, or from 0.1 to 0.15, or from 0.15 to 0.2, or from 0.2 to 0.25, or from 0.25 to 0.3.

### Lipid Nanoparticle Compositions

The nanoparticles produced by the method of the invention are nanoparticles comprising at least one amphiphilic lipid. In other words, the produced nanoparticles are lipid-based nanoparticles, or lipid nanoparticles. Examples of lipid nanoparticles may include nanoparticles which have complex internal structures, in some cases containing minimal to no aqueous interiors, as well as more structured nanoparticles such as liposomes, which term is typically used to refer to nanoparticles comprising at least one lipid bilayer, and optionally an aqueous-based interior phase. However, the terms “liposome” and “lipid nanoparticle” (sometimes abbreviated as LNP) are also often used interchangeably. Other expressions are often used for specific types of lipid nanoparticles, such as lipoplexes, lipopolyplexes, unilamellar liposomes, or multilamellar liposomes.

In one embodiment, the method according to the present disclosure produces a dispersion of nanoparticles selected from lipid nanoparticles, liposomes, lipoplexes or lipopolyplexes, and combinations thereof. In other embodiments, the lipid-based nanoparticles produced by the method of the invention are selected from the group of unilamellar liposomes (having a single bilayer), multilamellar liposomes, multi vesicular liposomes, single bilayer liposomes, double bilayer liposomes, lipid nanoparticles, lipoplexes, and lipopolyplexes. In some embodiments, the lipid-based nanoparticles produced by the method of the invention are any combination of the type of lipid nanoparticles listed above.

A skilled artisan would appreciate that a large number of lipid nanoparticles are currently used for clinical purposes or are in clinical development phases. Any of these nanoparticles can be produced by methods disclosed herein by impingement of all or part of their components in the organic and/or the aqueous solvent stream.

In some embodiments, the lipid nanoparticles comprise an amphiphilic lipid selected from the group of phosphatidylcholine, HSPC (hydrogenated soy phosphatidylcholine), a PEGylated lipid e.g. lipids conjugated to a PEG (polyethylene glycol) polymer, DSPE (distearoyl-sn-glycero-phosphoethanolamine), DSPC (distearoylphosphatidylcholine), DOPC (dioleoylphosphatidylcholine), DPPG (dipalmitoylphosphatidylglycerol), EPC (egg phosphatidylcholine), DOPS (dioleoylphosphatidylserine), POPC (palmitoyloleoylphosphatidylcholine), SM (sphingomyelin), lipids conjugated to MPEG (methoxy polyethylene glycol), DMPC (dimyristoyl phosphatidylcholine), DOTAP, DMPG (dimyristoyl phosphatidylglycerol), DSPG (distearoylphosphatidylglycerol), DEPC (dierucoylphosphatidylcholine), DOPE (dioleoly-sn-glycero-phophoethanolamine), Dipalmitoyl phosphatidylcholine (DPPC), lipids covalently bound i.e. conjugated to a methoxy polyethylene glycol (mPEG), mPEG-distearoyl phosphatidylethanolamine lipid conjugate, phospholipids covalently bound to mPEG, cardiolipin, DSPE-N-[amino(polyethylene glycol)-2000], or any combination thereof.

In some embodiments, the lipid nanoparticles comprise DPPC and cholesterol. In some embodiments, the lipid nanoparticles comprise HSPC:Cholesterol:PEG 2000-DSPE in a 56:39:5 molar ratio. In some embodiments, the lipid nanoparticles comprise DSPC and Cholesterol in a 2:1 molar ratio. In some embodiments, the lipid nanoparticles comprise DOPC, DPPG, Cholesterol and Triolein. In some embodiments, the lipid nanoparticles comprise EPC and Cholesterol in a 55:45 molar ratio. In some embodiments, the lipid nanoparticles comprise DOPS and POPC in a 3:7 molar ratio. In some embodiments, the lipid nanoparticles comprise SM and Cholesterol in a 60:40 molar ratio. In some embodiments, the lipid nanoparticles comprise DSPC, MPEG-2000, and DSPE in a 3:2:0.015 molar ratio.

In some embodiments, the lipid nanoparticles comprise DMPC and DMPG in a 7:3 molar ratio. In some embodiments, the lipid nanoparticles comprise HSPC, DSPG, Cholesterol, and an active ingredient (e.g. Amphotericin B) in a 2:0.8:1:0.4 molar ratio. In some embodiments, the lipid nanoparticles comprise Cholesteryl sulphate and Amphotericin B in a 1:1 molar ratio. In some embodiments, the lipid nanoparticles comprise DMPC and EPG in a 1:8 molar ratio. In some embodiments, the lipid nanoparticles comprise DOPC, DPPG, Cholesterol and Triolein. In some embodiments, the lipid nanoparticles comprise DEPC, DPPG, Cholesterol and Tricaprylin. In some embodiments, the lipid nanoparticles comprise DOPC and DOPE in a 75:25 molar ratio.

In some embodiments, the lipid nanoparticles comprise (4-hydroxybutyl)azanediyl)bis(hexane-6,1-diyl)bis(2-hexyldecanoate), 2-[(polyethylene glycol)-2000]-N,N-ditetradecylacetamide, 1,2-distearoyl-sn-glycero-3-phosphocholine (DSPC), and cholesterol. In some embodiments, the lipid nanoparticles comprise SM-102, polyethylene glycol [PEG], 2000-dimyristoyl glycerol [DMG], cholesterol, and 1,2-distearoyl-sn-glycero-3-phosphocholine [DSPC].

A skilled artisan would appreciate that the term “amphiphilic lipid” encompasses any lipid comprising hydrophilic and lipophilic properties, and that any of these amphiphilic lipids can be used in the methods discloses herein. In some embodiments, the amphiphilic lipid is selected from the group of fatty acids, glycerolipids, monoglycerides, diglycerides, glycerophospholipids, phospholipids, phosphatidylcholine, soybean phosphatidylcholine (SPC), phosphatidylethanolamine, phosphatidylserine, sphingolipids, sterols, cholesterol, prenols, carotenoids, retinol, retinal, retinoic acid, beta-carotene, tocopherol, saccharolipids, LIPOID S100, PEGylated lipids, 1,2-dimyristoyl-rac-glycero-3-methoxypolyethylene glycol (DMG-PEG), and combinations of these lipids. In one of the preferred embodiments, the amphiphilic lipid is, or comprises, a phospholipid.

In some embodiments, the amphiphilic lipid comprises a cationic lipid, an anionic lipid, or a neutral lipid, i.e. a lipid that carries a net positive charge, a net negative charge, or a net neutral charge, respectively, at about pH 3 to pH 9, in particular at a pH in the range from about pH 5 to pH 8. In some embodiments, an amphiphilic lipid comprises a PEGylated lipid, i.e. a lipid that is conjugated with a polyethylene glycol polymer.

In one embodiment, the nanoparticles comprise at least one cationic lipid, i.e. a lipid that carries a net positive charge at about pH 3-pH 9. The cationic lipid can be monocationic or polycationic. Any cationic amphiphilic molecule, e.g., a molecule which comprises at least one hydrophilic and lipophilic moiety is a cationic lipid within the meaning of the present invention. In one embodiment, the cationic lipid is an ionizable lipid. In one embodiment, at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof. The at least one cationic lipid contributes positive charges when complexing with negative charged active pharmaceutical ingredients, like e.g., nucleic acids.

In some embodiments, the lipid nanoparticles comprise an amphiphilic lipid such as a phospholipid and cholesterol.

The method according to the disclosure for producing the nanoparticles comprising at least one amphiphilic lipid includes a step of providing a first stream comprising an organic solvent and at least one said amphiphilic lipid. The first stream thus may comprise an organic solvent and at least one amphiphilic lipid such as any one of the amphiphilic lipids described herein, or combination of the classes or species of amphiphilic lipids as described herein.

In some embodiments, the first stream may comprise of an organic solvent and a combination of amphiphilic lipids. In one embodiment, the first stream comprises an organic solvent, and a cationic lipid, a phospholipid, a cholesterol, and a PEGylated lipid. Preferably, the first stream is a solution, i.e. liquid solution in which the lipid(s) are solubilized, or fully dissolved in the organic solvent, such as ethanol. In one specific embodiment, the first stream comprises ethanol, an ionizable cationic lipid, such as an amino lipid (e.g. DLin-KC2-DMA, or DLin-MC3-DMA), a cholesterol, a phospholipid and a PEGylated lipid (e.g. DMG-PEG). In some embodiments, the amount of cationic, or ionizable cationic lipid is at least 50 mol % relative to the total composition of lipids in the organic stream or solvent.

In some embodiments, the first stream may comprise of ethanol, and the lipids PEG2000-C-DMG (1,2-Dimyristoyl-rac-glycero-3-methoxypolyethylene glycol-2000), N,N-dimethyl-2,2-di-(9Z,12Z)-9,12-octadecadien-1-yl-1,3-dioxolane-4-ethanamine (DLin-KC2-DMA), 1,2-distearoyl-sn-glycero-3-phosphocholine (DSPC), and cholesterol. In some embodiments, the first stream comprises ethanol as the organic solvent, and the lipids DMG-PEG (1,2-Dimyristoyl-rac-glycero-3-methoxypolyethylene glycol-2000), (6Z,9Z,28Z,31Z)-heptatriaconta-6,9,28,31-tetraen-19-yl 4-(dimethylamino)butanoate (DLin-MC3-DMA), 1,2-distearoyl-sn-glycero-3-phosphocholine (DSPC), and cholesterol.

In other embodiments, the first stream or the second stream further comprises at least one further amphiphilic lipid. As understood herein, the at least one further amphiphilic lipid refers to a second, third, or fourth, et al. amphiphilic lipid in addition to the at least one amphiphilic lipid. Preferably, the organic solvent stream comprises the one or more amphiphilic lipids. The further amphiphilic lipid is preferably selected from the group of PEGylated lipids, cholesterol, ionizable cationic lipid, and phospholipids (like DSPC). In a specific embodiment, the first stream or the second stream comprises a PEGylated lipid at a concentration in the range of 0.1 mol % to 2 mol %. In one specific embodiment, the first stream comprises an organic solvent and a PEGylated lipid at a concentration in the range of 0.1 mol % to 2 mol %.

In some embodiments, the nanoparticles prepared according to the disclosed method further comprises at least one helper lipid. The helper lipid may be a neutral or an anionic lipid. In some embodiments, the helper lipid comprises a natural lipid, such as a phospholipid. In some embodiments, the helper lipid comprises an analogue of a natural lipid. In some embodiments, the helper lipid comprises a fully synthetic lipid. In some embodiments, the helper lipid comprises a lipid-like molecule, with no similarities with natural lipids. The helper lipid contributes to the stability of the lipid nanoparticle and to delivery efficiency. In specific embodiments, the helper lipid is selected from dioleoylphosphatidylethanolamine (DOPE), phosphatidylcholine, cholesterol, 1,2-dioleoyl-sn-glycero-3-phosphocholine (DOPC), PEGylating lipids, fatty acids and cholesteryl hemisuccinate (CHEMS), or analogs or derivates of these compounds. In some embodiments, the nanoparticles comprise a cationic lipid and a helper lipid selected from neutral or anionic lipids.

In some embodiments, the nanoparticle dispersion or nanoparticle comprises a single bilayer liposome. In some embodiments, the nanoparticle dispersion or nanoparticle comprises a double bilayer liposome. In some embodiments, the dispersion of nanoparticles comprises more than one type of liposome.

In some embodiments, the nanoparticle dispersion or nanoparticle comprising an amphiphilic lipid comprises a unilamellar liposomes. A skilled artisan would appreciate that an unilamellar liposome comprises a vesicle having a single phospholipid bilayer sphere enclosing an aqueous solution. In some embodiments, a nanoparticle comprises a multilamellar liposomes. A multilamellar liposome comprises a vesicle with an “onion” structure, i.e., a series of several unilamellar vesicles one inside the other, creating a multilamellar structure of concentric phospholipid spheres separated by layers of water.

In some embodiments, the nanoparticle dispersion produced according to the disclosed method is a dispersion of lipid nanoparticles comprising a nucleic acid. In some embodiments, the nanoparticle dispersion or nanoparticle comprises a lipoplex. A skilled artisan would appreciate that lipoplexes, sometimes called also lipopolyplexes, refer to complexes comprising nucleic acids encapsulated by lipidic vesicles. Lipopolyplexes may typically also comprise a polymer, in addition to the nucleic acid and lipid components. In some embodiments, the lipidic vesicles can be liposomes, i.e., a bilayered structure with an aqueous core comprising the nucleic acids. In some embodiments, lipoplexes comprise undefined complexes between lipids and nucleic acids. In some embodiments, lipoplexes can be formed by the electrostatic interaction between positively charged lipidic vesicles and the negatively charged nucleic acids.

In some embodiments, the nanoparticle dispersion or nanoparticle according to the present disclosure comprises a conventional liposome, a pH-sensitive liposome, a cationic liposome, a long circulating liposome, or an immuno-liposome.

### Active Pharmaceutical Ingredients (APIs)

In some embodiments, the nanoparticles comprise an active pharmaceutical ingredient (API). In some embodiments, in particular where the API is a small molecule that does not represent a nucleic acid, the API is dissolved or solubilized in the organic solvent of the first stream. In some embodiments, the API is dissolved or solubilized in the aqueous solvent of the second stream. In other words, the first stream provided under step a) of the method of the invention may further comprise an API. Alternatively, the second stream provided under step b) of the method of the invention further may comprise an API. In a specific embodiment, the nanoparticles comprise at least two different APIs that can be provided under step a) and/or step b). These embodiments provide flexibility when integrating active ingredients with different degrees of solubility.

In some embodiments, the API comprises a hydrophobic molecule. In some embodiments, the API comprises a hydrophilic molecule. In some embodiments, an API comprises an amphipathic molecule. In some embodiments, the API comprises a small molecule. In some embodiments, the API comprises a nucleic acid, or a derivative thereof. In some embodiments, the API may be a nucleic acid molecule which is, or comprises the genetic material for a vector, for example a viral vector (e.g. an adeno-virus associated vector) or a plasmid. In some embodiments, the API comprises a DNA molecule, or a derivative thereof, for example, plasmid DNA. In some embodiments, the API comprises an RNA molecule, or a derivative thereof. In a specific embodiment, the RNA molecule is selected from the group of mRNA, miRNA, pre-miRNA, saRNA, shRNA, siRNA, ribozyme, and antisense RNA. In a specific embodiment, the nucleic acid is antigen-coding, such as an antigen-encoding DNA or mRNA. In some embodiments, the API comprises a polypeptide, or a derivative thereof.

A skilled artisan would appreciate that a large number of APIs are available in the market or in development, and that any of these APIs can be used in the methods disclosed herein. In some embodiments, the API is selected from, but is not limited to, Zidovudine, Amphotericin B, Gentamycin, Polymixin B, Doxorubicin, Ketaconazole, Acetazolamide, N-Methyl-N-D-fructosyl amphotericin B methyl ester (MFAME), Ferrous sulphate, Hydroxyzine, Topotecan HCl, Daunorubicin, Cytarabine/Ara-C, Mifamurtide, Vincristine, Irinotecan, Amphotericin B, Verteporphin, Morphine sulfate, Bupivacaine, Inactivated hepatitis A virus (strain RGSB), Inactivated hemaglutinine of Influenza virus strains A and B, Amikacin, Tecemotide, T4 endonuclease V, Prostaglandin E-1 (PGE-1), Cisplatin, Platinum analogue cis-(trans-R,R-1,2-diaminocyclohexane) bis (neodecanoato) platinum (II), Semi-synthetic doxorubicin analogue annamycin, Lurtotecan, Potent topoisomerase I inhibitor, Irinotecan's active metabolite, Paclitaxel, All-trans retinoic acid, Mitoxantrone, Antisense oligodeoxynucleotide growth factor receptor bound protein 2 (Grb-2), Vinorelbine tartrate, Topotecan, PLK1 siRNA, PKN3 siRNA, CEBPA siRNA, Docetaxel, p53 gene, or Vinorelbine, or combinations thereof.

In some embodiments, the active ingredient is an RNA encoding an antigen, such as a RNA or mRNA for example encoding a coronavirus antigen. In some embodiments, the API comprises BNT162b2 RNA (BioNTech). In some embodiments, the active ingredient comprises mRNA-1273 (Moderna). In some embodiments, the API comprises ChAd0x1-SARS-CoV-2 (AstraZeneca).

As mentioned, the method according to the present disclosure comprises a step of providing a second stream comprising an aqueous solvent. The aqueous solvent can be a buffer system, a buffered aqueous solvent e.g. PBS, acetate buffer, citrate buffer, or TRIS buffer. Optionally, the aqueous solvent may include other excipients. In some embodiments, the aqueous solvent may comprise a cryoprotectant such as sucrose, trehalose, mannitol, sorbitol, glucose, fructose, proline, sorbitol, glycine betaine, polyethylene glycol, starch and dextran. The aqueous solvent may in some embodiments be a non-solvent to the API, when the API is provided in the first stream.

### Impingement Jet Reactors

As mentioned, the method of the invention involves the frontal collision of two streams, also referred to as jet impingement. As used herein, a stream means a stream of a fluid material, such as a liquid. In some embodiments, the nanoparticles disclosed herein are produced in an impinging jet reactor. Examples of such reactors are disclosed in EP1165224, EP 1352682, and US2017/0361299. A microjet reactor is an example of an impinging jet reactor.

Further, methods of solvent/nonsolvent precipitation by jet impingement are described in the art and disclosed for example in EP2395978.

An impinging jet reactor as used in the method of the invention comprises a reactor which is fed with a first stream comprising an organic solvent, and with a second stream comprising an aqueous solvent. Each stream is forced with raised pressure through a nozzle. The nozzles may have an opening in the range of 5-900 μm in diameter. Also preferred are diameters in the range from about 20 μm to about 800 μm.

The nozzles, which are herein termed also as pinholes, are located one opposite to the other, i.e. their pinholes are arranged such as to point at one another at an angle of about 180°. Thus, the injected streams collide frontally, i.e. at about 180° as well, and at high velocities in the mixing chamber of the reactor, which produces an intensive and turbulent mix generating a nanoparticle suspension. These nozzles, or pinholes create high-pressure jets that collide in a reactor chamber allowing for reduced or short mixing times.

To be more specific, according to method of the invention, the first stream comprising the organic solvent and the amphiphilic lipid is pumped under raised pressure through a first nozzle. The second stream comprising the aqueous solvent is pumped under raised pressure through a second nozzle. The raised pressure is provided by a first and a second pump as known the skilled artisan. The term “raised pressure” as used herein, can be used interchangeably with high pressure, or elevated hydrodynamic pressure, and refer to any pressure above the atmospheric pressure.

The first stream and the second stream are pumped into a reaction chamber where the two streams collide frontally. The reaction chamber, or reactor chamber, as used in parallel herein, provides a cavity for the two streams to form a highly turbulent mixing zone.

During collision of the first and the second stream, the streams may be considered as impinging liquid jets. In some embodiments, the hydrodynamic pressure for the impinging jets used during production are calculated on a theoretic basis and/or monitored using a pressure meter.

The hydrodynamic pressure calculation for the impinging streams of the first stream and the second stream can be calculated by Formula 1:

$$p = ( \frac{1}{2} * ρ * {[ \frac{Q}{π * {r}^{2}} ]}^{2} ) * 1 {0}^{- 5} \qquad Formula 1$$

in which
p=pressure in the stream ([bar] or [kPa], wherein 1 bar is 100 kPa)
ρ=density of the composition (all constituents) of the stream ([kg/m³])
Q=flow rate of the stream ([m³/s])
r=radius of pin hole ([m]).

The stream velocity v [m/s] can be calculated according to Formula 2:

$$v = {[ \frac{Q}{π * {r}^{2}} ]}^{2} \qquad Formula 2$$

In some embodiments, flow rates are measured by implementation of flow meters in the feeding lines to the impingement jet reactor (e.g. a microjet reactor), so that the flow can also be confirmed during the process.

In some embodiments, the present disclosure provides for a method for producing a dispersion of nanoparticles comprising at least one amphiphilic lipid, wherein the method comprises the steps of: a) providing a first stream comprising an organic solvent and the amphiphilic lipid; b) providing a second stream comprising an aqueous solvent; c) pumping the first stream under a raised pressure through a first nozzle and pumping the second streams under a raised pressure through a second nozzle into a reaction chamber; wherein the first nozzle is located at an angle of about 180° from the second nozzle; d) colliding the first stream and the second streams frontally in a reaction chamber; wherein the flow rate ratio between the first stream and the second stream is in a range from 1:1.5 to 1:4.5; and wherein the method comprises the use, or the provision of a impingement jet reactor, comprising said first and second nozzle, and reaction chamber.

In some embodiments, the present disclosure provides for a method for producing a dispersion of nanoparticles comprising at least one amphiphilic lipid, wherein the method comprises the steps of providing a jet impingement reactor comprising a first nozzle and a second nozzle, and a reaction chamber, wherein the first nozzle is located at an angle of about 180° from the second nozzle;
a) providing a first stream comprising an organic solvent and the amphiphilic lipid;
b) providing a second stream comprising an aqueous solvent;
c) pumping the first stream under a raised pressure through the first nozzle and pumping the second streams under a raised pressure through the second nozzle into a reaction chamber;
d) colliding the first stream and the second streams frontally (at an angle of about 180°) in a reaction chamber;
wherein the flow rate ratio between the first stream and the second stream is in a range from 1:1.5 to 1:4.5.

In some embodiments, the impingement jet reactor provided in the method according to the present disclosure has at least two nozzles (pin-holes) located opposite one another (at an angle of about 180° C. from each other). Each first and second nozzle may be connected with a feedline, wherein optionally, each feedline is associated with a pump arranged for providing, at raised pressure, the first stream and the second stream according to the embodiments or combination of embodiments described herein, to the respective nozzles. These streams may collide at a common collision point in a reactor chamber, which optionally may be enclosed by a reactor housing. In an optional embodiment, another opening is provided in the reactor housing through which optionally, a gas, an evaporating liquid, a cooling liquid, or a cooling gas can be passed to maintain the gas atmosphere in the reactor chamber or for cooling. In a further embodiment, such opening is absent A further opening is provided for removing the resulting products and optionally excess gas from the reactor chamber. In the embodiment where a gas, an evaporating liquid or a cooling gas is introduced into the reactor chamber through an opening in order to maintain a gas atmosphere inside the reactor or to cool the resulting products, the resulting products and excess gas may be removed from the reactor chamber through this opening by overpressure on the gas inlet side or by under-pressure on the product and gas outlet side. Use of solvent/nonsolvent precipitation may be carried out in such an impingement jet reactor to obtain a dispersion of precipitated lipid-based nanoparticles as described in accordance with any one or combination of the embodiments described herein.

In some embodiments, the jet impingement reactor used, or provided in the method according to the present invention comprises a reaction chamber defined by an interior surface of a reaction chamber wall which has a substantially spheroidal overall shape, as described in more detail below. The reaction chamber is further characterised in that it comprises (a) a first and a second fluid inlet, wherein the first and the second fluid inlet are arranged at opposite positions of a first central axis of the reaction chamber such as to point at one another, and wherein each of the first and the second fluid inlet comprises a nozzle; and (b) a fluid outlet arranged at a third position, said third position being located on a second central axis of said chamber, the second central axis being perpendicular to the first central axis. Moreover, the distance between the nozzle of the first fluid inlet and the nozzle of the second fluid inlet is the same or smaller than the diameter of the reaction chamber along the first central axis.

Substantial improvements over conventional jet reactors may be achieved by such reactor, in particular based on the substantially spheroidal overall shape of the reaction chamber and its small size in particular as reflected by a relatively short distance between the fluid inlet nozzles. Without wishing to be bound by theory, it is believed that the spheroidal overall shape eliminates some of the detrimental effects of irregularly shaped reaction chambers known in the art that have internal angles, edges or corners, and the associated dead volume zones. The small size and minimised distance between the fluid inlet nozzles is believed to intensify the turbulent mixing of fluids in the chamber and facilitate the proper alignment of the nozzles on the same axis, such as to achieve a frontal collision of the two fluids that are injected into the chamber by the nozzles.

As used herein, a substantially spheroidal overall shape means that at least the larger part of reaction chamber as defined by the internal surface of the chamber wall has the shape of a sphere or is similar to a sphere. For example, the spheroid may be shaped such that some of its cross sections are ellipses. In one embodiment, all parts or portions of the reaction chamber or of the interior surface of the chamber wall except for those portions that hold or define an inlet or an outlet opening are substantially spheroidal, or even spherical.

In case the outlet opening, which is typical relatively large in diameter compared to the diameter of the nozzles or inlet openings, is understood as a deviation from the otherwise spherical shape of the reaction chamber, the shape of the reaction chamber may also be described as a spherical cap, also referred to as a spherical dome. In a preferred embodiment, such spherical cap has a height, a basis, and a radius along the first central axis (i.e. on which the two fluid inlets are positioned), wherein the height is larger than said radius, and wherein the basis is defined by the fluid outlet. In other words, the spherical dome formed by the reaction chamber is larger in volume than a corresponding hemisphere, which also means that diameter of the outlet opening is smaller than the largest diameter of the reaction chamber. In a specific embodiment, the height of the dome is in the range of about 110% to about 170% of the radius. For example, the height may be about 120% to about 160% of the radius, such as about 120%, about 130%, about 140%, about 150%, or about 160% of the radius.

Another preferred feature of the reactor which may be used in accordance with the method according to the present disclosure relates to the arrangement of the nozzles. As mentioned, each of the first and the second fluid inlet comprises a nozzle, and in the assembled state of the reactor, the distance between the nozzle of the first fluid inlet and the nozzle of the second fluid inlet is the same or smaller than the diameter of the reaction chamber along the first central axis. This is in contrast to some jet reactors known in the art which have nozzles that are retracted. In a preferred embodiment of the invention, the nozzles—more precisely their downstream ends—are neither retracted nor do they protrude into the reaction chamber, but they are substantially aligned with the interior surface of the reaction chamber wall.

It is further preferred that the reaction chamber is provided with a rather small internal volume which would also correspond to a small distance between the nozzles if arranged according to the preferences explained above. As used herein, the distance between the first nozzle and the second nozzle should be understood as the distance between the downstream ends (i.e., the ends of the nozzles that point to the centre of the reaction chamber). Preferred reaction chamber volumes are below about 0.5 mL, and preferred distances between the nozzles are below about 7 mm. In one specifically preferred embodiment, the reaction chamber has a volume of not more than about 0.25 mL and the distance between the nozzle of the first fluid inlet and the nozzle of the second fluid inlet is not more than 5 mm. In further preferred embodiments, the volume of the reaction chamber is not more than about 0.2 mL, for example about 0.15 mL, and the distance between the two nozzles is not more than about 4 mm. Still smaller dimensions may also be useful. For clarity, it should be noted that for the purpose of providing these preferences with respect to the volume of the reaction chamber, the respective values have been calculated under the assumption that the reaction chamber has a substantially spherical shape irrespective of the outlet opening. In other words, the outlet opening has not been interpreted as forming the base of a spherical cap that is smaller in volume than the sphere that it is derived from. If the outlet opening were to be understood as being planar such as to form the base of a spherical segment that represents the volume of the reaction chamber, the values in mL provided above should be adapted accordingly, taking the dimensions of the outlet opening into consideration.

In one embodiment, the reaction chamber is free of other inlet or outlet openings. In other words, the first and a second fluid inlet and the fluid outlet represent the only openings of the reaction chamber that are provided in the chamber wall. This is also in contrast to other embodiments of jet impingement reactors which may exhibit one or more additional inlets, such as an inlet for a gas to be introduced to the reaction chamber or an outlet for degassing purposes. It may be advantageous to not have additional inlets or outlets which may also negatively interfere with the impingement process and result in uncontrolled precipitation or the building up of contamination in such additional openings. Such reactor may bring about the advantage of better control over the interaction and mixing of the first fluid stream with the second fluid stream, improved cleanability and increased batch-to-batch consistency.

As used herein, a reactor having a reaction chamber with one or more additional inlet or outlet openings that are inactivated by a closure mechanism should also be understood a reactor whose reaction chamber has no further inlet or outlet opening beyond the two essentially required inlet openings for the first and the second fluid and the outlet opening for the fluid that results from the mixing (and/or reaction) of the first and the second fluid streams in the reaction chamber.

In accordance with the basic concept of a jet impingement reactor, in some embodiments, the reactor should preferably be configured and/or arranged such as to direct a first fluid (i.e. first stream comprising an organic solvent and amphiphilic lipid) and a second fluid (i.e. second stream comprising an aqueous solvent) into the reaction chamber in such a way that the two fluids impinge on, or collide frontally, with one another. This is particularly relevant for a precise positioning and orientation of the nozzles that are comprised in the two fluid inlets. In a preferred embodiment, accordingly, the jet impingement reactor is characterised in that the nozzles of the first and second fluid inlet are arranged such as to direct a first and a second fluid stream along the first central axis towards the centre of the chamber and to allow the first fluid stream and the second fluid stream to collide at an angle of about 180°. As used herein, the collision at an angle of about 180° may also be referred to as a frontal collision. In this context, the expression “about” means that the actual angle is sufficiently close to 180° to ensure that the collision of the first liquid stream and the second liquid stream results in a rapid and highly turbulent fluid flow in the mixing zone, such that thorough mixing takes place within an extremely short time, e.g., typically within a matter of milliseconds.

In some embodiments, the jet impingement reactor is equipped with exchangeable nozzles. This will speed up product and process development efforts as it allows a quick screening of process parameters using the same reactor. This is different from prior art reactors which typically have non-removable or non-replaceable nozzles, i.e., nozzles that are glued, welded, crimped or thermofitted in such a way that they cannot be disconnected from the reactor in a non-destructive manner, so that the testing of certain process parameters, in particular the testing of different nozzle diameters, would require the use of several reactors within the respective series of experiments. As used herein, a nozzle diameter should be understood as the internal diameter of the nozzle opening, which may also be referred to as pinhole size or diameter in case the nozzle is a plain orifice nozzle. In other words, this embodiment brings about a substantially increased versatility of the reactor.

In one embodiment, each of the first and the second fluid inlet is provided by a fluid inlet connector having an upstream end, a downstream end holding the nozzle of the first or second fluid inlet, and a fluid conduit for conducting a fluid from the upstream end to the downstream end; wherein the downstream end of each fluid inlet connector is reversibly insertable into the chamber wall such as to provide the first and the second fluid inlet. According to this embodiment, the nozzles are exchangeable in that reversibly insertable inlet connectors holding the nozzles are provided. The nozzles may be firmly affixed to the exchangeable connectors. As used herein, an inlet connector (or fluid inlet connector) may be any piece having an upstream end and a downstream end and an internal fluid conduit configured to provide a fluidic connection between the upstream end and the downstream end.

An advantage of such reactor configuration with exchangeable fluid inlet connectors (and thereby exchangeable nozzles) is the reactor exhibits better cleanability and a reduced cycle time. A further advantage of a reactor having exchangeable nozzles or fluid inlet connectors which may readily be replaced, substantially increases the versatility of the reactor, including the possibility to work with different nozzle diameters using one and the same reactor chamber.

In one embodiment, the fluid inlet connectors are affixed to the chamber wall by means of a releasable compression fitting. For example, the fluid inlet connectors that provides the first and/or the second fluid inlet is affixed to the chamber wall by means of a single ferrule fitting or a double ferrule fitting. Other tight fittings that are capable of preventing leakage under high pressures are also useful to the extent that they are releasable.

In one preferred embodiment, the reactor comprises a fluid inlet connector that has (i) an upstream segment comprising the upstream end of the fluid inlet connector and an upstream portion of the fluid conduit; and (ii) a downstream segment comprising the downstream end of the fluid inlet connector with the nozzle and a downstream portion of the fluid conduit, wherein the diameter of the upstream portion of the fluid conduit is larger than the diameter of the downstream portion of the fluid conduit. In this context, the diameter should be understood as the internal diameter.

The downstream portion may be shaped as, or provided by, a capillary tube whose diameter is substantially smaller than that of the upstream portion. For example, in one embodiment, the diameter of the downstream portion is not larger than half the diameter of the upstream portion. In another embodiment, the diameter of the downstream portion is about 40% of the diameter of the upstream portion or less. Optionally, the upstream portion may be substantially longer than the downstream portion. For example, the ratio of the length of the upstream portion to the length of the downstream portion may be 5:1 or higher, or even 8:1 or higher.

In one specific embodiment, the downstream end of the fluid inlet connector is externally cone-shaped, and the chamber wall exhibits a corresponding void that is also cone-shaped and dimensioned such as to receive the downstream end of the fluid inlet connector. Using this configuration facilitates the insertion of the fluid inlet connector into the reactor and at the same time the proper alignment of the respective nozzle on the first central axis as described above. Preferably, the reactor is equipped with two fluid inlet connectors having basically the same overall configuration, except that their nozzles may have different diameters. The fluid inlet connectors as described herein represent an aspect of the present disclosure.

The nozzles may be of any type or geometry that allows the injection of the first and the second fluid into the reaction chamber in the form of a fluid stream, using an appropriate pressure.

In one of the preferred embodiments, the nozzle of the first and/or the second fluid inlet is a plain-orifice nozzle. For this embodiment, it is further preferred that both nozzles are plain-orifice nozzles. As used herein, a plain-orifice nozzle is a nozzle that characterised by a simple orifice that essentially has the shape of a simple (i.e. substantially cylindrical) through-hole, which may in view of its small dimensions also referred to as pinhole. Alternatively, the nozzle may also be provided as a shaped-orifice nozzle, as long as the selected shape results in the generation of a fluid stream that is capable of frontally colliding with a second fluid stream in the reaction chamber at the respective working pressures.

If a plain-orifice nozzle is used, such nozzle may be provided as a piece made of a particularly hard material, such as sapphire, ruby, diamond, ceramic, glass (such as borosilicate glass) or steel. In the case of steel, it is preferred that a steel quality having a high hardness and low abrasiveness is used, such as high-speed steel (HSS), which is an alloy steel containing carbide-forming elements such as tungsten, molybdenum, chromium, vanadium, and cobalt, the total amount of alloy elements typically being in the range of about 10-25 wt. %, or tungsten steel, also referred to as hard alloy, in which tungsten and cobalt are the main alloy elements.

If sapphire, ruby or diamond nozzles are used, these may be prefabricated, inserted into the downstream end of the downstream portion of the fluid inlet connector and affixed, e.g. by crimping. The nozzles' tolerances that depend on the prefabrication methods should be taken into consideration. If nozzles of steel are used, it is useful to prepare the entire fluid inlet connector or at least the downstream portion thereof from the respective steel quality and then introduce the required orifices. In this manner, the alignment of the nozzles with the first central axis may be further improved.

The diameters of the nozzles, i.e. of the orifices of the nozzles, are typically in the range of below about 1 mm. As process development in the field of pharmaceutics often involves the use of very costly materials, in particular in the development of nanoparticular forms of novel chemical entities, new biological drugs, highly specialised colloidal carrier systems for advanced therapeutics and the like, the volumes of liquids used for process development should be minimised. This is best achieved, inter alia, with even smaller nozzles, such as nozzles having orifices of 0.5 mm or less in diameter.

Accordingly, it is one of the preferred embodiments of the invention that a jet impingement reactor as described above is characterised in that the nozzle of the first fluid inlet has a first orifice diameter and the nozzle of the second fluid inlet has a second orifice diameter, and in that the first orifice diameter and/or the second orifice diameter are in the range of 20 μm to 500 μm. Preferably, both the first orifice diameter and the second orifice diameter are in the range of 20 μm to 500 μm, or in the range of about 50 μm to 500 μm. Also preferred are reactor configurations in which at least one of the orifice diameters is about 500 μm, about 400 μm, about 300 μm, about 200 μm, about 100 μm, about 50 μm, or about 20 μm, respectively. Even smaller diameters, e.g. below 20 μm, may be considered.

In some embodiments, the diameters of the first and the second nozzle (i.e. nozzle orifice or opening) are the same, such as about 300 μm, about 200 μm, about 100 μm. In a more preferred embodiment of the reactor used in the present disclosure, the reactor has two nozzles that differ in size. In other words, the one of the orifice diameters is larger than the other orifice diameter, according to this further preferred embodiment. Such asymmetric nozzle configuration may be advantageous in various ways: for example, it may be used to minimise the introduction of a solvent that is required for processing purposes but undesirable in the final product. It may also be used for the generation of two liquid streams that have different flow rates but similar kinetic energy as they are injected through the nozzles into the reaction chamber where they collide.

In one embodiment, the diameter of the second nozzle (i.e. its orifice) is at least 20% larger than that of the first nozzle. In a further embodiment, the ratio of the second orifice diameter to the first orifice diameter is from about 1.2 to about 5. For example, the following nozzle pairs may be used, wherein the first value represents the approximate diameter of the second orifice and the second value the approximate diameter of the first orifice: 100 μm and 50 μm; 200 μm and 100 μm; 200 μm and 50 μm; 300 μm and 200 μm; 300 μm and 100 μm; 300 μm and 50 μm; 400 μm and 300 μm; 400 μm and 200 μm; 400 μm and 100 μm; 400 μm and 50 μm; 500 μm and 400 μm; 500 μm and 300 μm; 500 μm and 200 μm; 500 μm and 100 μm; 500 μm and 50 μm. Again, these pairs are non-limiting examples, and other orifice diameter combinations may also be useful, depending on the specific product or process.

Moreover, the inventors have found that it is useful to observe certain dimensional relationships in the configuration of the reactor, in particular when small nozzles are used. As already mentioned, it is preferred that the reaction chamber is small, generally speaking. It was also found that it is useful for some processes to provide the reactor with a reaction chamber diameter that is not more than 100 times the diameter of the nozzle orifices or, if nozzles with different sizes are used, with a chamber diameter that is not more than about 100 times the diameter of the larger nozzle's orifice diameter. For example, if the larger nozzle has an orifice diameter of 100 μm, it is preferred according to this specific embodiment that the diameter of the reaction chamber is about 10 mm or less.

In a related embodiment, the ratio of the diameter of the reaction chamber along the first central axis to the second orifice diameter is in the range from 6 to 60. For example, if the diameter of the second orifice is about 200 μm, the diameter of the reaction chamber along the first central axis would be in the rage from about 1.2 mm to about 12 mm, according to this specific embodiment. However, reactors equipped with larger nozzles may require other dimensional considerations.

According to a further related embodiment, the ratio of the diameter of the reaction chamber along the first central axis to the diameter of the fluid outlet is in the range of about 1.2 to about 3. For example, a reaction chamber having a diameter of about 3 mm would have an outlet diameter of about 1 mm to about 2.5 mm, according to this specific embodiment.

When selecting the outlet diameter, also the nozzle orifice diameters should be taken into consideration. For example, small nozzle sizes (i.e. orifices) such as below 100 μm should be combined with a small fluid outlet diameter, such as below 1 mm, in order to ensure that the pressure in the reaction chamber is sufficiently high to support turbulence and rapid mixing of the two fluids. For example, when using two nozzles with orifices of 50 μm, a fluid outlet diameter of 0.5 mm may be used. Based on the disclosure and guidance provided above, it would be clear for the skilled person that further variations of the dimensional factors may also be useful to accommodate certain product or process requirements.

With respect to the material of the reactor, in particular the material of the chamber wall, various types of sufficiently hard and wear-resistant materials may be used. In some of the embodiments, the reaction chamber wall (3) is made of a material selected from metal, glass, glass-ceramics, ceramics, and thermoplastic polymers. An example of a useful metal is stainless steel. In one of the preferred embodiments, the reactor of the invention comprises a reaction chamber wall made of stainless steel. Carbides and coated alloys may also be used, depending on the type of product for whose manufacture the reactor is to be used. Moreover, it is also preferred that the interior surface of the chamber wall exhibits a smooth finish. A smooth finish may be characterised by a low Ra value that expresses the surface roughness. The Ra value represents the arithmetic mean roughness value from the amounts of all values when measuring the surface along a surface profile. According to one of the preferred embodiments, the interior surface of the reaction chamber wall exhibits a surface roughness of not more than 0.8 Ra, wherein Ra is determined according to ISO 4287:1997.

In an alternative embodiment, the jet impingement reactor provided according to the method of the present disclosure comprises a reaction chamber wall made of a thermoplastic polymer, or a material comprising a thermoplastic polymer, such as a mixture of thermoplastic polymers or a mixture of a thermoplastic polymer and an additive, such as colouring agents, antioxidants, antistatics, glass fibres and the like. An advantage of a reaction chamber wall made of a thermoplastic polymer or a materials based on a thermoplastic polymer is that the reactor may potentially be manufactured by injection moulding, which is a very cost-effective manufacturing method. Examples of potentially suitable thermoplastic polymers include, without limitation, polytetrafluoroethylene (PTFE), polyamide, polycarbonate (PC), polyether ether ketone (PEEK), polyethylene (PE), polypropylene (PP), polystyrol (PS), acrylonitrile butadiene styrene (ABS), polyoxymethylene (POM), polyphenylsulfone (PPSF or PPSU), and polyetherimide (PEI). In one of the embodiments, the thermoplastic polymer is selected from PTFE and PEEK.

In some embodiments, the jet impingement reactor comprises (i) a reaction chamber wall made of, or comprising, a thermoplastic polymer, and (ii) fluid inlet nozzles (i.e. the nozzles of the first and the second fluid inlet) made of a material selected from metal, glass, glass-ceramic, and ceramic. A particular advantage of such embodiment is that it combines nozzles having a high degree of hardness and strength while at the same time allowing the main body of the reactor, i.e. the reaction chamber wall, to be cost-effectively manufactured by injection moulding. In these embodiments, the fluid inlet nozzles may either be arranged in an exchangeable or non-exchangeable manner with respect to the reaction chamber wall. If designed to be exchangeable, this results in the advantage that the jet impingement reactor is versatile and can be used with a high degree of flexibility for various products and processes. On the other hand, if designed to be non-exchangeable, this brings about the advantage of very cost-effective manufacture in that nozzles may be pre-fabricated and then inserted into the respective mould for, or during the injection moulding process by which the main body of the reactor, i.e. at least the reaction chamber wall, is produced. Further details regarding the method such as the temperature at which the molten thermoplastic polymer may be injected into the mould depend on the selected material, i.e. the nature of the thermoplastic polymer, and are generally known to those skilled in the art.

In a further aspect, the invention provides a method based on the use of the reactor described in detail above. In particular, the invention discloses a method of mixing two fluids, the method comprising the steps of: (i) providing the jet impingement reactor as described above; (ii) directing a first fluid stream through the first fluid inlet into the reaction chamber; (iii) directing a second fluid stream through the second fluid inlet into the reaction chamber such as to collide with the first fluid stream at an angle of about 180°.

As used herein, a fluid is a liquid or gaseous material that continually flows or deforms when it is subjected to shear stress. Preferably, the two fluids mixed according to the invention are liquid materials, such as liquid solutions, suspensions or emulsions, and most preferably liquid solutions. As used herein, the mixing of the two fluids in the reactor may optionally further involve other physical or chemical changes beyond the mere mixing, such as precipitation, emulsification, complexation, self-assembly, or even chemical reactions; but all these optional processes are triggered by the mixing of the two liquids as achieved by the use of the jet impingement reactor according to the invention.

Operating the reactor under jet impingement conditions typically involves the selection of appropriate nozzle sizes as described above, and the providing of the two fluid streams at a pressure or flow rate that causes the fluids to be injected through the nozzles into the reaction chamber towards its centre where they ideally collide frontally.

If the reactor is configured to have exchangeable fluid inlet connectors that are reversibly insertable into the chamber wall such as to provide the first and the second fluid inlet, the method step of providing the jet impingement reactor may comprise the sub-steps of (i) selecting a first fluid inlet connector having a first nozzle and a second fluid inlet connector having a second nozzle; and (ii) inserting the first fluid inlet connector and the second fluid inlet connector into the chamber wall such as to provide a jet impingement reactor having a first and a second fluid inlet. As explained above, the orifice diameters may differ between the first and the second nozzle.

In one of the preferred embodiments of the method, the first fluid stream comprises an organic solvent and at least one amphiphilic lipid, and the second fluid stream is an aqueous solvent, which may act as a non-solvent or antisolvent for the at least one, or more amphiphilic lipids, so that the collision and mixing of the two streams in the reaction chamber leads to the precipitation or self-assembly of the nanoparticles (e.g. lipid nanoparticles or liposomes) comprising the amphiphilic lipid. In one of the preferred embodiments, the first and the second fluid stream are forced through the respective fluid inlet nozzles at a pressure in the range of about 0.1 to about 120 bar. In this context, and unless the context dictates otherwise, the pressure is expressed as gauge pressure, i.e., the overpressure, or pressure difference to the ambient (atmospheric) pressure that is typically obtained from a pressure gauge that is in fluid connection with the respective fluid to be measured. In a further preferred embodiment, the first and the second fluid stream are forced through the respective fluid inlet nozzles at a pressure in the range of about 1 to about 40 bar.

In a further preferred embodiment, each of the first and the second fluid stream is directed into the reaction chamber at a flow rate in the range of about 1 to 1000 mL/min. In this context, the flow rates are provided for each individual stream, unless indicated otherwise. Other preferred ranges of the flow rate are from about 2 to 1000 mL/min, from 5 to 1000 mL/min, from about 5 to about 500 mL/min and from about 10 to about 300 mL/min, respectively.

Like the preferences regarding the pressures, the preferred flow rates should also be understood as generally applicable and thus combinable with one another. In other words, there is also an option or even preference for an embodiment of the method in which the first and the second fluid are directed through the respective nozzles into the reaction chamber at a pressure in the range of about 0.1 to about 120 bar, in particular of about 1 to about 40 bar, at a flow rate in the range of about 10 to 300 mL/min.

As described above, according to one of the preferred embodiments of the jet impingement reactor, the two nozzles may differ in pinhole size, i.e., the orifice of the first nozzle may be larger, or smaller than that of the second nozzle. In a related embodiment, the method of the invention is performed with such reactor equipped with two different nozzles. Alternatively, or in addition, flow rate of the second stream, also referred to a second fluid stream may be larger than the flow rate of the first (fluid) stream. In a further preferred embodiment, the method is characterised in that (i) the orifice of the second nozzle is larger than the orifice of the first nozzle; and/or (ii) the flow rate of the second fluid stream is larger than the flow rate of the first fluid stream; and wherein the pressure of the second fluid stream and of the first fluid stream is adapted such as to cause the first fluid stream and the second fluid stream to have substantially the same kinetic energy when entering the reaction chamber.

In this context, the kinetic energy may optionally be calculated according to the formula

$${E}_{k} = 1 / 2 * m * {v}^{2}$$

wherein m is the mass of the stream per volume unit and v is the speed of the stream.

The advantage of working with two fluid streams that have a similar or even substantially the same kinetic energy is that the collision point in a spheroidal (i.e. symmetric) reaction chamber is at or close to the centre of the chamber. Thus, it is possible to better control the impingement process and rule out the impact of uncontrolled collision points which may have various unknown or even undesirable effects on the process. Preferably, the kinetic energy of the fluid streams are sufficiently similar to cause a collision or impingement of the streams at or near the centre of the reaction chamber.

### Methods for Producing Nanoparticle Suspension

It was unexpectedly found herein that, under certain conditions, parameters such as flow rate ratio, pressure in the stream, and pin hole radius, affect the characteristics of the nanoparticles produced by a jet impingement reactor.

According to one aspect of the invention, disclosed herein are methods to produce a dispersion of nanoparticles, said nanoparticles comprising desired size and PDI, by methods comprising impinging two streams at specific flow rate ratios (FRR). Some specific FRR were useful for producing nanoparticles of desired characteristics, as for example small size and low PDI.

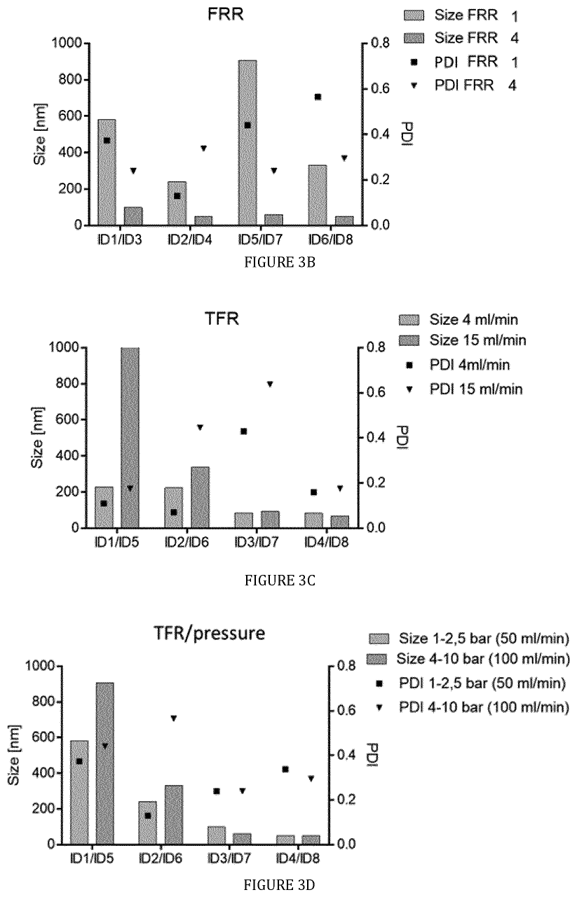

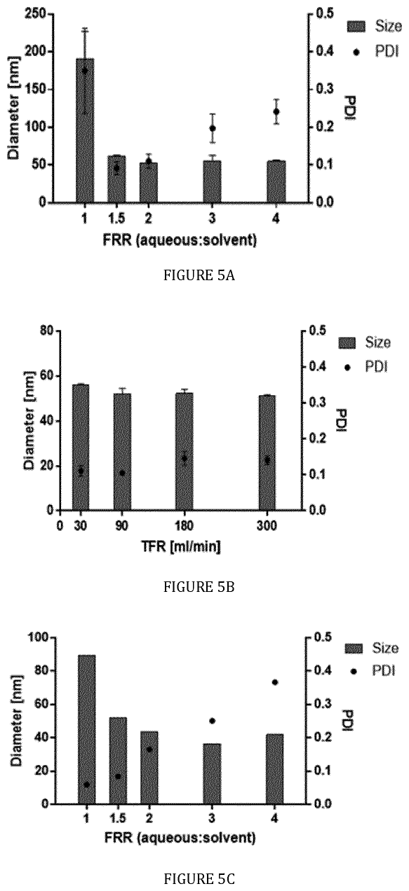

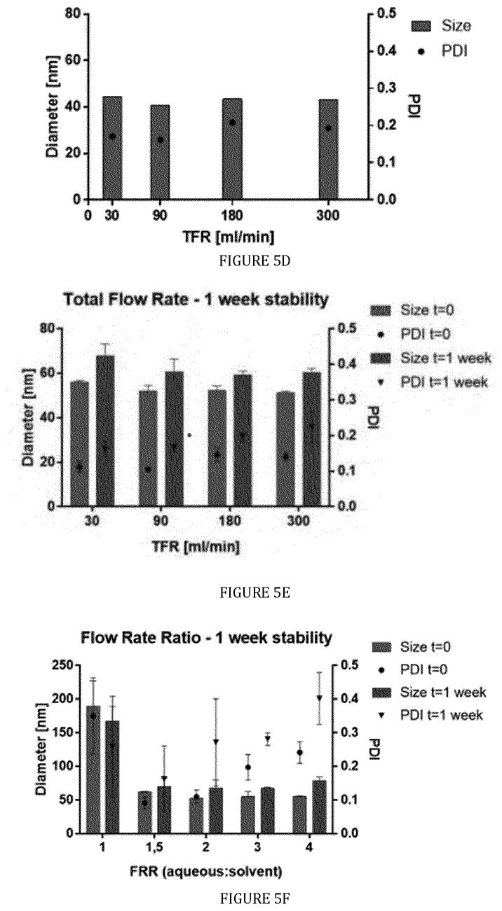

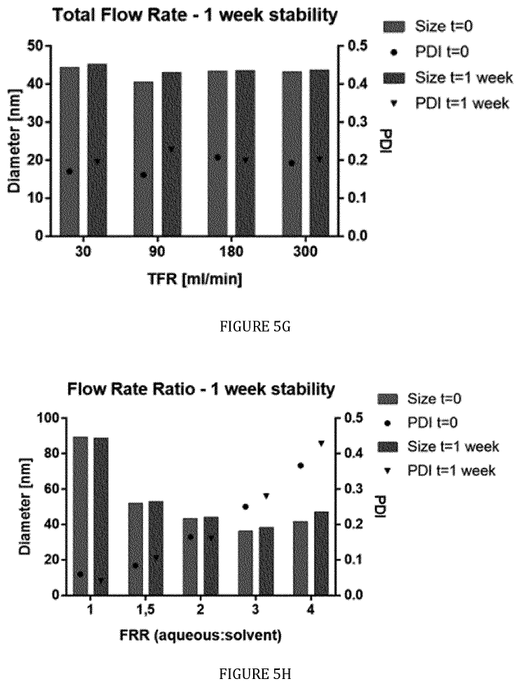

According to first aspect of the invention, the flow rate ratio between the first stream (organic solvent) and the second stream (aqueous solvent) is in a range from about 1:1.5 to about 1:4.5. It was surprisingly found that, when this ratio is applied, the method provides a robust and stable process and reproducibly yields particles that are stable and of good quality. It was observed by the inventors that flow rate ratios outside this range, such as 1:6 or 1:8, had a negative impact on the polydispersity index (PDI) of liposomes and LNPs. In a preferred embodiment, the flow rate ratio between the first stream and the second stream is in a range from about 1:1.5 to about 1:4, or from about 1:2 to about 1:4. It could be shown that these flow rate ratios are particularly advantageous for obtaining lipid-based nanoparticles having a small average particle size, such as below 80 nm, or below 60 nm, respectively, as can be seen in FIGS. 3B and 5A. In some embodiments, the flow rate ratio between the first stream and the second stream is in a range from 1:1.5 to 1:3, more preferably from 1:1.5 to 1:2.5, more preferably from 1:1.5 to 1:2. In further embodiment the flow rate ratio between the first stream and the second stream is about 1:1.5, 1:2, 1:2.5, 1:3, 1:3.5, or 1:4.

Further, disclosed herein are methods to produce a dispersion of nanoparticles, said nanoparticles comprising desired size and PDI comprising impinging two streams at specific total flow rates (TFR). In general, the TFR is in the range from about 0.1 ml/min to about 2,000 ml/min.

In one of the preferred embodiments, the TFR is selected in the range from about 1 ml/min to about 1,000 ml/min, or from about 5 ml/min to about 800 ml/min, or from about 10 ml/min to about 800 ml/min, or from about 25 ml/min to about 800 mL/min or from 30 ml/min to about 800 ml/min.

It could be found that, when applying the flow rate ratios according to the method of the invention, the production of the nanoparticles was unaffected by the total flow rate of the first and the second stream, e.g. in terms of size and stability of the nanoparticles. Thus, a robust process for the production of a dispersion of nanoparticles at different scales or output rates is provided with the invention.

It was surprisingly found that, under certain conditions, nanoparticles of desirable characteristics, such as small size and low PDI, could be obtained by the methods disclosed herein even at a high TFR, such as a TFR of more than about 200 ml/min. For example, a TFR in the range from about 200 ml/min to about 1,000 ml/min may be useful in situations in which process efficiency and high productivity are important. In one particular embodiment, the TFR is in the range from about 200 ml/min to about 800 ml/min. In another specific embodiment, the TFR is from 280 ml/min to 320 ml/min, such as about 300 ml/min.

A skilled artisan would appreciate that the total flow rate (TFR) refers to the total volumes injected into the reaction chamber per time unit. In other words, the total flow rate is the sum of the flow rate of the first stream and the second stream.

In some embodiments, the total flow rate, i.e. the sum of the flow rates of the first stream and the second stream, is low, such as below about 200 ml/min, or even below about 100 ml/min. For example, the TFR may be in the range from about 1 ml/min to 200 ml/min, or from about 5 ml/min to 200 ml/min, or from 10 ml/min to 200 ml/min, from 15 ml/min to 200 ml/min, or from about 1 ml/min to about 100 ml/min, or from about 5 ml/min to about 80 ml/min. Such low TFR may be useful in situations in which expensive starting materials are used for small batches.

In some embodiments, the total flow rate, i.e. the sum of the flow rates of the first stream and the second stream, is in the range of from 30 ml/min to 320 ml/min, preferably from 100 ml/min to 320 ml/min, most preferably from 280 ml/min to 320 ml/min. In a more specific embodiment, the total flow rate is in the range of from 280 ml/min to 320 ml/min, more preferably from 290 ml/min to 310 ml/min. In some embodiments, the TFR is between about 20 ml/min and about 500 ml/min, or between about 50 ml/min and about 200 ml/min, or between 50 ml/min and 100 ml/min.

In a specific embodiment, the total flow rate is in the range of from 100 ml/min to 500 ml/min and the flow rate ratio between the first stream and the second streams is in a range from 1:1.2 to 1:2.5. In a more specific embodiment, the total flow rate is about 300 ml/min and the flow rate ratio between the first stream and the second streams is 1:2. These combinations of parameters (flow rate ratio and high total flow rate) yielded in the production of nanoparticles with advantageous properties, e.g. with an average size below 60 nm and an PDI below 0.25.

In some embodiments, the TFR is about 30 ml/min, or about 40 ml/min, or about 50 ml/min, or about 60 ml/min, or about 70 ml/min, or about 80 ml/min, or about 90 ml/min, or about 100 ml/min.

In some embodiments, the TFR is about 150 ml/min, or about 200 ml/min, or about 250 ml/min, or about 300 ml/min, or about 320 ml/min, or about 350 ml/min.

As shown by Formula 1 above, the stream pressure is regulated, among others, by the size of the nozzles openings. In some embodiments, the size of the first and/or second nozzle opening is smaller than 100 μm in diameter. In some embodiments, the size of a nozzle opening is between 100 and 200 μm radius. In some embodiments, the size of a nozzle opening is between 200 and 300 μm radius. In some embodiments, the size of a nozzle opening is between 300 and 400 μm radius. In some embodiments, the size of the nozzle opening is between 400 and 500 μm radius. In some embodiments, the size of the nozzle opening is between 500 and 600 μm radius. In some embodiments, the size of the nozzle opening is between 600 and 700 μm radius. In some embodiments, the size of the nozzle opening is larger than 200 μm radius.

In some embodiments, the size of the first and/or second nozzle opening is about 100 μm radius. In some embodiments, the size of a nozzle opening is about 200 μm radius. In some embodiments, the size of a nozzle opening is about 300 μm radius. In some embodiments, the size of a nozzle opening is about 400 μm radius. In some embodiments, the size of a nozzle opening is about 500 μm radius. In some embodiments, the size of a nozzle opening is about 600 μm radius. In some embodiments, the size of a nozzle opening is about 700 μm radius. In some embodiments, the size of a nozzle opening is about 800 μm radius. In some embodiments, the size of a nozzle opening is about 900 μm radius. In some embodiments, the size of a nozzle opening is about 1000 μm radius.

In some embodiments, the radius of the first and second openings are the same. In some embodiments, the radius of the first and the second openings are different, or asymmetric. In some embodiments, the radius of said second opening is 100 μm and the radius of said first opening is 100, 200, 300, 400, or 500 μm. In some embodiments, the radius of the second opening is 200 μm and the radius of said first opening is 100, 200, 300, 400, or 500 μm. In some embodiments, the radius of said second opening is 300 μm and the radius of said first opening is 100, 200, 300, 400, or 500 μm.

In some embodiments, the radius of said second opening is 400 μm and the radius of said first opening is 100, 200, 300, 400, or 500 μm. In some embodiments, the radius of said second opening is 500 μm and the radius of said first opening is 100, 200, 300, 400, or 500 μm.

In some embodiments, the radius of said first and second openings are 300 μm and 200 μm, respectively. In some embodiments, the radius of said first and second openings are 400 μm and 200 μm, respectively. In some embodiments, the radius of said first and second openings are 500 μm and 200 μm, respectively.

In some embodiments, the first nozzle comprises a first opening and the second nozzle comprises a second opening, wherein the first opening and the second opening have a radius in the range of 200 to 500 μm.

In another embodiment of the method according to the present disclosure, the first nozzle comprises a first opening and the second nozzle comprises a second opening, wherein the first opening and the second opening have a diameter in the range of 40 μm to 800 μm. In one embodiment, the first nozzle comprises a first opening and the second nozzle comprises a second opening, wherein the first opening and the second opening have a diameter in the range of 200 to 500 μm.

In some embodiments, the diameter of the first and second openings may be the same In other embodiments, the diameter of the first and second openings are different, or asymmetric. In some embodiments, the diameter of said first opening is 100 μm and the diameter of said second opening is 100, 200, 300, 400, or 500 μm. In some embodiments, the diameter of the first opening is 200 μm and the diameter of said second opening is 100, 200, 300, 400, or 500 μm. In some embodiments, the diameter of said first opening is 300 μm and the diameter of said second opening is 100, 200, 300, 400, or 500 μm.

In a specific embodiment, the diameter of the first opening is different from the diameter of the second opening. In one embodiment, the diameter of the first or second opening is at least about 50% larger than the diameter of the other opening. In one embodiment, the diameter of the second opening may be larger than the diameter of the first opening. In one of the preferred embodiments, the diameter of the second opening is at least 50% larger than the diameter of the first opening. In another embodiment, the diameter of the second opening is from about 1.5 times (50%) to about 5 times larger than the diameter of the first opening.

In some embodiments, the diameter of said first and second openings are 500 μm. As disclosed above, the first and second streams are pumped through a first and a second nozzle at a raised pressure. Said pressure can be calculated from the flow rate and density of the streams and from the radius of the pin holes through which the streams are ejected, as shown in Formula 1 above.

A skilled artisan would appreciate that the terms “raised pressure”, “high pressure”, “overpressure” or “hydrodynamic pressure”, which are used herein interchangeably, refer to any pressure above the atmospheric pressure. Further, as used herein in some embodiments, the pressure of the first and second stream is measured in comparison to the atmospheric pressure. Therefore, a pressure of 0.1 bar is to be understood as 0.1 bar above the atmospheric pressure.

In some embodiments, the pressure of the first and the second streams are similar. In some embodiments, the pressure of the first stream is higher than that of the second stream. In some embodiments, the pressure of the first stream is lower than or equal to that of the second stream.

In some embodiments, the pressure of the first stream is lower than about 30 bar, lower than about 25 bar, lower than about 12 bar, or lower than about ≤5 bar. In some embodiments, the pressure in the first stream is from about 0.0001 bar to about 30 bar, from about 0.001 bar to about 25 bar, from about 0.01 bar to about 12 bar, or from about 0.01 bar to about 5 bar.

In some embodiments, the pressure of the second stream is lower than about 30 bar, lower than about 25 bar, lower than about 12 bar, or lower than about ≤5 bar. In some embodiments, the pressure in the second stream is from about 0.0001 bar to about 30 bar, from about 0.001 bar to about 25 bar, from about 0.01 bar to about 12 bar, or from about 0.01 bar to about 5 bar.

In some embodiments, the pressure of said first or second stream is lower than 0.1 bar. In some embodiments, the pressure of the first or second streams is about 0.1, 0.2, 0.3, 0.4, 0.5, 0.6, 0.7, 0.8, 0.9, or 1 bar. In some embodiments, the pressure of the first or second streams is about 2, 3, 4, 5, 6, 7, 8, 9, or 10 bar. In some embodiments, the pressure of the first or second streams is about 10, 15, 20, 25, or 30 bar. In some embodiments, the pressure of the first or second streams is higher than 30 bar.

In some embodiments the pressure of the first and/or second stream is between 0.1 to 45 bar. In some embodiments, the pressure of the first stream and/or second is between 0.1 and 15 bar.

A skilled artisan would appreciate that in some instances it is helpful calculating and using a total pressure, which is herein defined as the sum of the pressures of the first and the second streams.

Based on the mathematic relation of hydrodynamic pressure and jet velocity, the jet velocity of the first stream and second streams can be controlled or determined by nozzle opening radius. In some embodiments, the jet velocity is below 70, 50, 25, or 10 m/s. In some embodiments, the jet velocity of the first and second streams are different.

In some embodiments, the first stream comprises an organic solvent. In some embodiments, the organic solvent is a solvent for the amphiphilic lipid, the API, or both. A skilled artisan would appreciate that many organic solvents are used in the pharmaceutical industry. Any of them can be used in the methods disclosed herein. In some embodiments, the organic solvent comprises an alcohol, a ketone, a halogenated solvent, an amide, or an ether. In some embodiments, an organic solvent is selected from the group comprising ethanol, ethylene, bromide, butanol, acetone, chloroform, 2-ethylhexanol methylethylketone, ethylene chloride, isobutanol, methylisobutylketone, dichloromethane, isopropanol, methylisopropylketone, tetrachloroethylene methanol, mesityl oxide, carbon tetrachloride, propanol, trichloroethylene, propylene glycol, 1,4-dioxane butyl ether, ethyl ether, dimethylformamide, diisopropyl ether, dimethyl sulfoxide, tetrahydrofuran, tert-butyl methyl ether, hydrocarbons, aromatic hydrocarbons, cyclohexane, toluene, hexane, and xylene.

In some embodiments, the organic solvent is miscible with water. In some embodiments, the organic solvent comprises a mixture of 2 or more organic solvents. In some embodiments, the organic solvent is an alcohol. In some embodiments, the organic solvent comprises ethanol. In some embodiments, the organic solvent comprises acetone.

In some embodiments, the second stream comprises an aqueous solvent. In some embodiments, the aqueous solvent is a non-solvent for the amphiphilic lipid, for the API, or for both. A skilled artisan would appreciate that many aqueous solvents are used in the pharmaceutical industry. Any of them can be used in the methods disclosed herein. In some embodiments, the aqueous solvent comprises a phosphate buffer saline (PBS), or Dulbecco's phosphate-buffered saline (DPBS). In some embodiments, the pH of the aqueous solvent is modified to any desired value.

### Further Aspects

In some embodiments, the method further comprises a step of purifying the nanoparticle dispersion by filtration; subjecting the nanoparticle dispersion to lyophilization; or removing the organic solvent from the nanoparticle dispersion by cross-flow filtration.

In some embodiments, the nanoparticle dispersion is subjected to sterile filtration. In some embodiments, the filter material is selected from cellulose acetate, regenerated cellulose, polyamide, polyethersulfone (PES), modified polyethersulfone (mPES), polyvinylidene fluoride (PVDF) and polytetrafluoroethylene (PTFE). A skilled artisan would appreciate that sterile filtration allows parenteral application of the product and is method of choice for products for parenteral use that cannot be terminally sterilized by moist heat or dry heat. In some embodiments, the nanoparticle dispersion is subjected to lyophilization. Lyophilization may increase stability of the lipid-based nanoparticle (e.g. liposome or LNP) dispersion.

In some embodiments, the organic solvents used for production of the nanoparticle dispersion can be removed via a cross-flow filtration process. In one embodiment, the organic solvents used for production of the lipid-based nanoparticle (e.g. liposome or LNP) dispersion are removed via a cross-flow filtration process.

In a further aspect, disclosed herein is a nanoparticle dispersion produced by the method of the first aspect of the invention. In one embodiment, the average particle size of the nanoparticles ranges from 20 nm to 100 nm, or from 30 nm to 90 nm, such as from 40 nm to 80 nm, when measured with Dynamic Light Scattering.

In a further aspect, disclosed herein is a pharmaceutical composition comprising a nanoparticle dispersion produced by the method of the first aspect of the invention. In some embodiments, the pharmaceutical composition is intended for parenteral or oral application. In some embodiments, the pharmaceutical composition is a lyophilisate for reconstitution before application. In some embodiments, the pharmaceutical composition can be terminally sterilized by filter sterilization.

Further embodiments comprised in the present disclosure are comprised in the following list of numbered items:
1. A method for producing a dispersion of nanoparticles comprising at least one amphiphilic lipid, wherein the method comprises the steps of:
   a) providing a first stream comprising an organic solvent and the amphiphilic lipid;
   b) providing a second stream comprising an aqueous solvent;
   c) pumping the first stream under a raised pressure through a first nozzle and pumping the second streams under a raised pressure through a second nozzle into a reaction chamber; wherein the first nozzle is located at an angle of about 180° from the second nozzle;
   d) colliding the first stream and the second streams frontally in a reaction chamber; and
wherein the flow rate ratio between the first stream and the second stream is in a range from 1:1.5 to 1:4.5.
2. The method according to item 1, wherein the flow rate ratio between the first stream and the second stream is in a range from 1:1.5 to 1:4, or from 1:2 to 1:4.5, or from 1:2 to 1:4, or wherein the flow rate ratio between the first stream and the second stream is about 1:1.5, 1:2, 1:2.5, 1:3, 1:3.5, 1:4 or 1:1.45.
3. The method according to item 1 or 2, wherein the total flow rate is in the range of from 1 ml/min to 1,000 ml/min, or from 5 ml/min to 800 ml/min.
4. The method according to one of the items 1 to 3, wherein the total flow rate is in the range of from 100 ml/min to 500 ml/min and wherein the flow rate ratio between the first stream and the second streams is in a range from 1:1.2 to 1:2.5.
5. The method according to any of the items 1 to 4, wherein the first nozzle comprises a first opening and the second nozzle comprises a second opening, wherein the first opening and the second opening have a diameter in the range of 40 μm to 800 μm.
6. The method according to item 5, wherein the diameter of the first or second opening is at least about 50% larger than the diameter of the other opening.
7. The method according to any of items 1 to 6, wherein the pressure of the first stream is lower than or equal to the pressure of the second stream.
8. The method of any of the items 1 to 7, wherein the nanoparticles are selected from the group consisting of unilamellar liposomes, multilamellar liposomes, single bilayer liposomes, double bilayer liposomes, multivesicular liposomes, lipid nanoparticles, lipoplexes, polyplexes, and solid lipid nanoparticles.
9. The method according to any of the items 1 to 8, wherein the amphiphilic lipid is selected from the group of fatty acids, glycerolipids, monoglycerides, diglycerides, glycerophospholipids, phospholipids, phosphatidylcholine, soybean phosphatidylcholine (SPC), phosphatidylethanolamine, phosphatidylserine, sphingolipids, sterols, cholesterol, prenols, carotenoids, retinol, retinal, retinoic acid, beta-carotene, tocopherol, saccharolipids, LIPOID S100, PEGylated lipids, and 1,2-dimyristoyl-rac-glycero-3-methoxypolyethylene glycol (DMG-PEG).
10. The method according to any of the items 1 to 9, wherein the first stream or the second stream further comprises at least one further amphiphilic lipid.
11. The method according to any of the items 1 to 10, wherein the first stream or the second stream comprise a PEGylated lipid at a concentration in the range of 0.1 mol % to 2 mol %.
12. The method according to any of the items 1 to 11, wherein the first or the second streams further comprise an active pharmaceutical ingredient.
13. The method according to item 12, wherein the active pharmaceutical ingredient is selected from the group of small molecules, peptides, and nucleic acids.
14. The method according to any of the items 1 to 13, further comprising a step of purifying the nanoparticle dispersion by filtration;
   subjecting the nanoparticle dispersion to lyophilization; or
   removing the organic solvent from the nanoparticle dispersion by cross-flow filtration.
15. A nanoparticle dispersion produced by a method according to any of items 1 to 14, wherein the average particle size of the nanoparticles is preferably from 20 nm to 100 nm.

### EXAMPLES

The following examples are presented in order to more fully illustrate some embodiments of the technology disclosed herein. They should in no way be construed, however, as limiting the scope of the invention. The examples disclose a stepwise approach with increasing complexity, aimed to show the suitability of the impinging jet technology disclosed herein for the manufacture of liposomes and lipid nanoparticles (LNPs).

Features of the equipment systems and reactors used for the experiments disclosed herein are listed in Table 1. System A (or apparatus A, or equipment A) is intended as small-scale system for batch-wise or continuous production with flow rates from 0.1 ml/min up to 60 ml/min, depending on the reactor. System B is adapted for batch-wise or continuous production with higher total flow rates of up to about 500 ml/min. A commercial microfluidic system (NanoAssemblr® Ignite™, Precision NanoSystems, Vancouver, Canada) as a small-scale precipitation system with laminar flow mixing was used as comparison to the turbulent mixing-based jet impingement reactor systems A and B. For clarity, the comparator system does not cause two streams to collide frontally, as in a jet impingement reactor.

TABLE 1
Overview of the equipment used.

| Equipment | Comparative System | System A | System B |
|---|---|---|---|
| Reactor | Cartridge micromixer | Jet impingement reactor | Jet impingement reactor |
| | (NanoAssemblr ® | according to | according to |
| | Ignite ™, staggered | EP 1 165 224 B1 | EP 1 165 224 B1 |
| | herringbone mixer) | | |
| Principle of mixing | Laminar flow | Turbulent mixing | Turbulent mixing |
| Typical pinhole | — | 40-300 | 100-500 |
| range (μm) | | | |
| Intended use | Development stage (lab | Development stage | Development |
| (scale) | scale) | (lab scale) | (pilot scale) |
| Volume of typical | 1-10 ml | 1-10 ml | 50 ml-1,000 ml |
| batch | | | |

### Example 1—Production of Liposomes Comprising Soy-Phosphatidylcholine and Cholesterol

Methods: To demonstrate the usefulness of the jet impingement technology for the manufacturing of lipid-based nanoparticles, a formulation using soy-phosphatidylcholine (SPC) and cholesterol was selected.

A total of 10 experiments were performed using the equipment described herein above as System B at different flow rate ratios (FRR), total flow rates (TFR), using different nozzle opening diameters and at different SPC/cholesterol ratios.

In addition, 8 experiments on the comparator system, the Ignite equipment from Precision NanoSystems were performed, to compare the two systems. Samples were analyzed for average particle size and polydispersity index (PDI) by dynamic light scattering (DLS) using a Malvern Zetasizer Nano ZS.

A solution consisting of soybean phosphatidylcholine (SPC) and cholesterol in ethanol was prepared and used as a first (organic) liquid stream. A total lipid concentration of 4 mg/ml in ethanol was used. An aqueous buffer (PBS) at pH 7.4 was used for the second (aqueous) liquid stream. The total flow rates tested in System B were 50 ml/min and 100 min/ml. Flow rate ratios (abbreviated as FRR) of the first (organic) stream:second (aqueous) stream of 1:1 and 1:4 were tested. SPC:cholesterol molar ratios of 3:1 and 2:1 were tested. The parameters used in the experiments are summarized in Table 2A The parameters used in the experiments on the comparator equipment are summarized in Table 2B and are analogous in respect of the FRR and tested formulations; only differing in TFR due to scale differences with System B.

TABLE 2A
Parameters of experiments using System B

| | | | | | | FRR | |
|---|---|---|---|---|---|---|---|
| | Pinhole | | | | | (first organic | |
| | diameters | Calculated | | | | stream:second | weight ratio |
| ID | (first/second) | pressure | | TFR | | aqueous stream) | SPC:cholesterol |
| 1 | 200/200 μm | 1-2 | bar | 50 | ml/min | 1:1 | 3:1 |
| 2 | 200/200 μm | 1-2 | bar | 50 | ml/min | 1:1 | 2:1 |
| 3 | 100/200 μm | 2-4 | bar | 50 | ml/min | 1:4 | 3:1 |
| 4 | 100/200 μm | 2-4 | bar | 50 | ml/min | 1:4 | 2:1 |
| 5 | 200/200 μm | 4-6 | bar | 100 | ml/min | 1:1 | 3:1 |
| 6 | 200/200 μm | 4-6 | bar | 100 | ml/min | 1:1 | 2:1 |
| 7 | 100/200 μm | 12-14 | bar | 100 | ml/min | 1:4 | 3:1 |
| 8 | 100/200 μm | 12-14 | bar | 100 | ml/min | 1:4 | 2:1 |
| 9 | 200/300 μm | <1 | bar | 50 | ml/min | 1:4 | 3:1 |
| 10 | 200/300 μm | <1 | bar | 50 | ml/min | 1:4 | 2:1 |

TABLE 2B
Parameters of experiments using laminar flow reactor system

| ID | TFR | | FRR | SPC/cholesterol ratio |
|---|---|---|---|---|
| 1 | 4 | ml/min | 1:1 | 3:1 |
| 2 | 4 | ml/min | 1:1 | 2:1 |
| 3 | 4 | ml/min | 1:4 | 3:1 |
| 4 | 4 | ml/min | 1:4 | 2:1 |
| 5 | 15 | ml/min | 1:1 | 3:1 |
| 6 | 15 | ml/min | 1:1 | 2:1 |
| 7 | 15 | ml/min | 1:4 | 3:1 |
| 8 | 15 | ml/min | 1:4 | 2:1 |

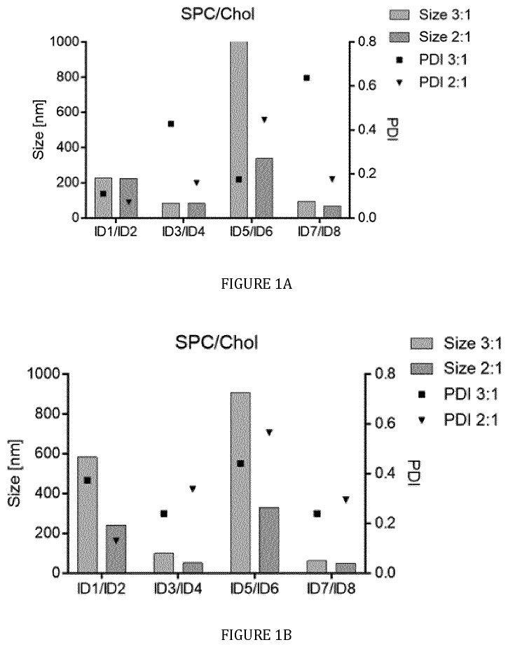

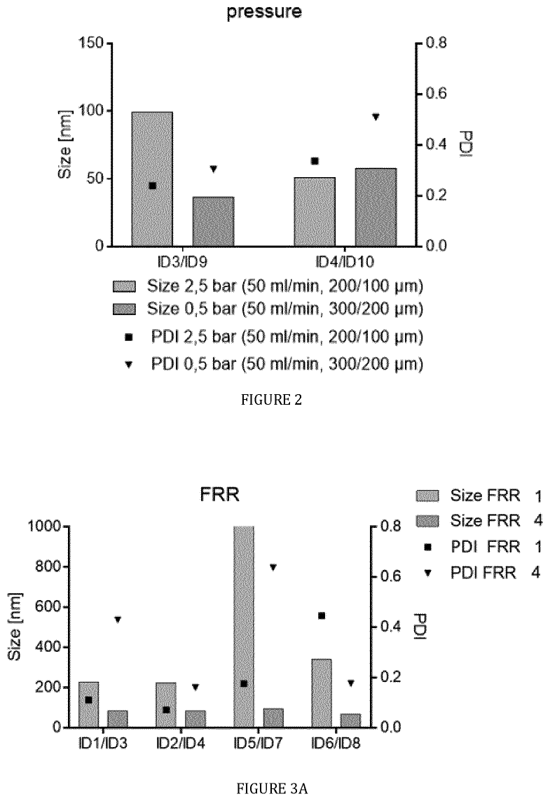

Results: Comparable results in terms of particle sizes and PDI of the liposomes as measured by dynamic light scattering) were obtained using jet impingement reactor System B and the commercial laminar flow reactor system (NanoAssemblr® Ignite™), demonstrating the feasibility of using of a jet impingement reactor and turbulential flow mixing to obtain unloaded liposomes and moreover the use of pilot-scale equipment. The results obtained using the respective systems are shown in FIGS. 1 to 3.

### Example 2—Production of PEGylated Liposomes

### Methods

Equipment System B as described in Table 1 above comprising a jet impingement reactor was used to prepare PEGylated liposomes, comprising a PEGylated lipid, a phosphatidyl choline, and cholesterol.

Two organic liquids comprising soybean phosphatidylcholine (SPC), cholesterol, and 1,2-dimyristoyl-rac-glycero-3-methoxypolyethylene glycol (DMG-PEG) at a molar ratio of 36.2%, 63.2%, and 0.6%, respectively (formulation 1); and at a molar ratio of 52%, 45%, and 3%, respectively (formulation 2), in both cases dissolved in ethanol, were prepared and either of them was used as the first stream. In both cases, the total lipid concentration in the organic solution was 6 mg/ml. PBS pH 7.4 was used as aqueous liquid for the second stream.

Particles were produced at a total flow rate (TFR) of 30, 90, 180, or 300 ml/min, and at a flow rate ratio of the first (organic) stream to the second (aqueous) stream (FRR) of 1:1, 1:1.5, 1:2, 1:3, or 1:4. All samples were prepared using an impingement jet reactor with nozzle diameters of 200 (first nozzle) and 300 μm (second nozzle). Samples were analyzed for particle size and polydispersity index (PDI) by dynamic light scattering (DLS), and selected samples were analyzed by cryo-TEM. All samples produced according to the invention were diluted to 5% EtOH content immediately after production and before DLS analysis

Results: Liposomes of uniform size distribution and low PDI (<0.2) were obtained for both of the tested formulations.

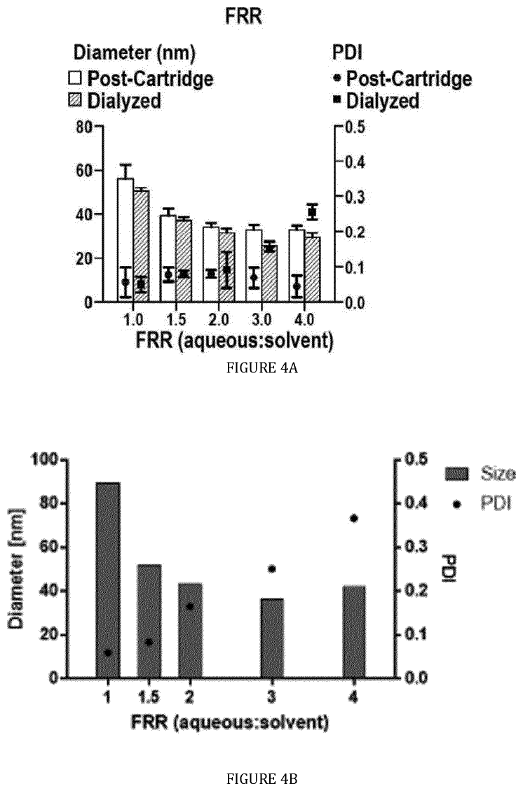

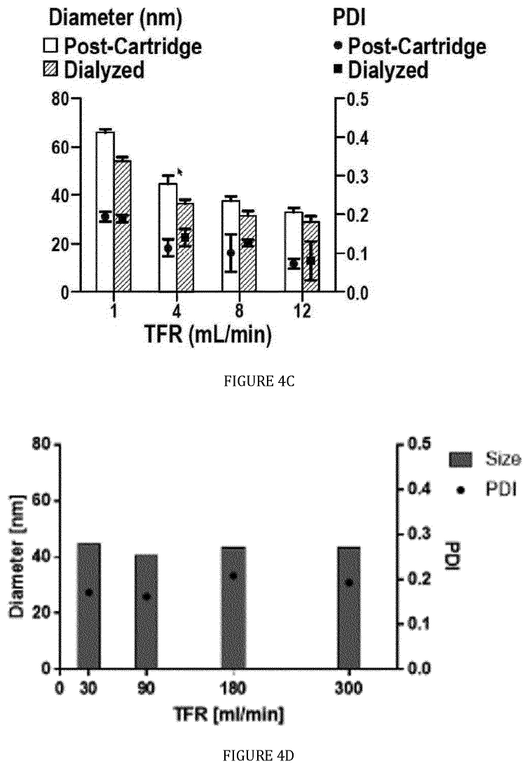

The characteristics of liposomes obtained with formulation 2 were compared to published data of liposomes with the similar composition which were generated with a microfluidic mixer system based on laminar flow (see Brown A., Thomas A., Shell Ip, Heuck G., Ramsay E., Liposomes, published by Precision NanoSystems Inc. 2018 (https://www.precisionnanosystems.com/docs/default-source/pni-files/app-notes/pni-app-bt-013.pdf?sfvrsn=aa668324_0 retrieved 05.05.2021); which are represented in FIGS. 4A and 4C. According to the published data, the effect of increasing flow rate of the second (i.e. aqueous stream) compared to the first (i.e. organic) stream using this type of equipment and mixing resulted in decreasing particle size and a trend towards increasing PDI. Increasing the TFR in this microfluidic system also led to decreases in particle size.

It was observed, similar to the laminar flow mixing system, that the effect of lower FRRs when using the impingement jet method of the invention also led to smaller particle sizes and a trend towards larger PDI values (see FIG. 4B). However, and in comparison, no influence of TFR on particles size and PDI of the liposomes was observed when using the impingement jet method of the invention (see FIG. 4D). Particle sizes were observed to remain consistent over a TFR ranging from 30 ml/min to 300 ml/min. This indicates that a highly turbulent mixing process, as achieved by jet impingement, i.e. frontal collision of the first and second streams, may be favourable to achieve a robust, and scalable process, thus providing a versatile process technology which can be easily adapted to various production scenarios, including, for example. the preparation of multiple batches of liposomes or lipid nanoparticles at low or high volumes as needed.

The liposomes prepared from the two different lipid formulations using SYSTEM B equipment were characterized (see FIGS. 5A to 5F). Qualitatively similar trends for FRR and TFR can be observed for both formulations. At a FRR of 1 the formulation with lower PEG-lipid content (formulation 1) showed the highest PDI (0.35), while for the formulation with higher PEG-lipid concentration (formulation 2) the lowest PDI (0.06). Furthermore, the effect of decreasing FRR (the ratio between the flow rate of the first i.e. organic stream to the flow rate of the second, i.e. aqueous stream) on the particle size is more prominent for the formulation with lower PEG-lipid content.

The formulation with higher PEG-lipid concentration showed a higher physical stability after 1 week (FIGS. 5G and 5H), underlining the stabilizing effect of PEG-lipids. It can be concluded that both the basic particle properties like the limit size as well as the responsiveness to process parameters are determined by the liposomal composition. Generally, higher PEG-lipid concentrations lead to smaller and more stable particles, which were less influenced by process parameters.

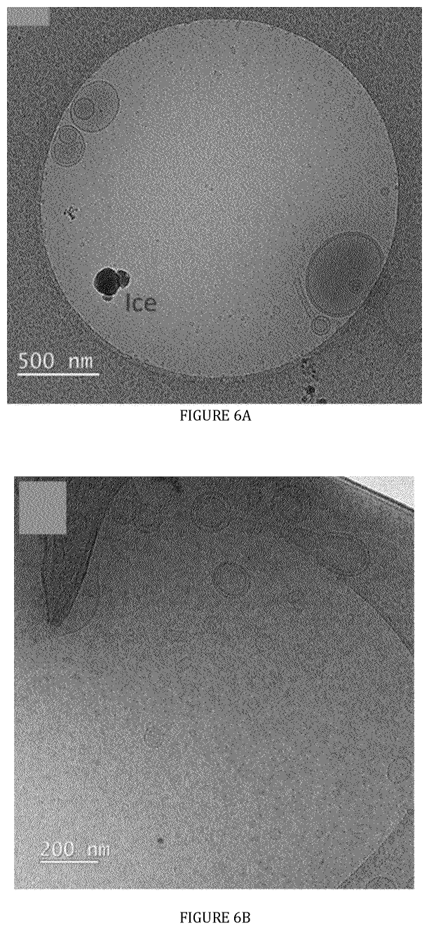

Cryo-TEM analysis showed that the produced liposomes were predominantly unilamellar or bilamellar (FIGS. 6A and 6B), and the DLS particle size results were confirmed. In most cryo-TEM images, also larger particles (up to μm range) with various morphologies were observed. These larger particles can be removed by filtration (e.g. 0.2 μm) before cryo-TEM analysis if needed. The results indicate that process conditions such as larger TFRs and associated higher total pressures lead to higher numbers of bilamellar liposomes and to a more heterogeneous morphology.

### Example 3—Lipid Nanoparticles Design of Experiment

To verify the suitability of the jet impingement process for the production of lipid nanoparticles (LNPs) and to identify critical process parameters that affect the process and outcome of LNP production using the SYSTEM B equipment as outlined above, a statistical design of experiment (DoE) approach was conducted. In brief, in a DoE with D-optimal design (Design Expert v12 software) the process parameters TFR, FRR and 3 different reactors (200/300, 200/400, 500/500 μm nozzle size) were varied and their impact on particle size, PDI and morphology evaluated upon dialysis with PBS. The test formulation was based on the composition of Onpattro® (patisiran), except that no siRNA was used (DLin-MC3/Cholesterol/DSPC/DMG-PEG2000 with a molar ratio of 50/38.5/10/1.5 with total lipid concentration of 10 mg/mL in EtOH).

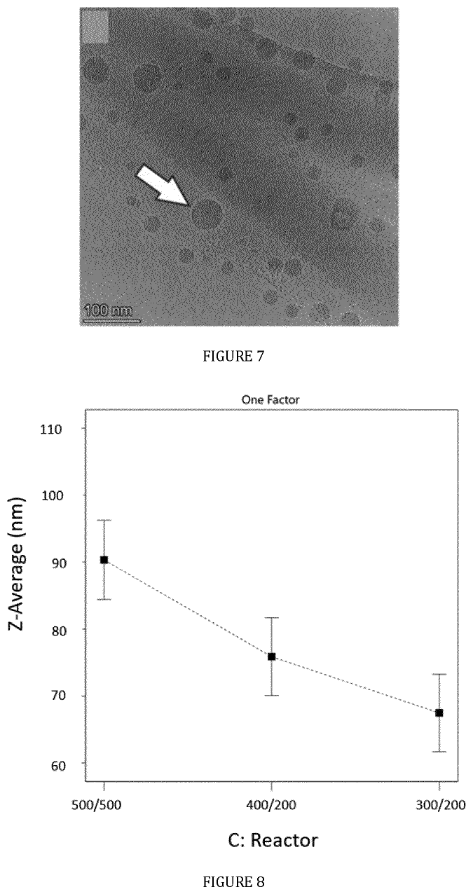

All 20 samples in the DoE contained unloaded LNPs with a particle size range of 64-106 nm (PDI: 0.12-0.25), as measured by DLS and cryo-TEM analysis (see sample in FIG. 7). This was in line with the particle size range of 60-100 nm of Onpattro® as mentioned in a public assessment report.

These results suggest that the jet impingement process is suitable to produce high quality (unloaded) LNPs using a placebo LNP formulation, even at high output rates, produced at a total flow rates of up to about 300 ml/min. All samples retained their particle size and PDI after prolonged storage for one week at refrigeration conditions (+4° C.), further indicating that the process of the invention is suitable for LNP production of the used formulation.

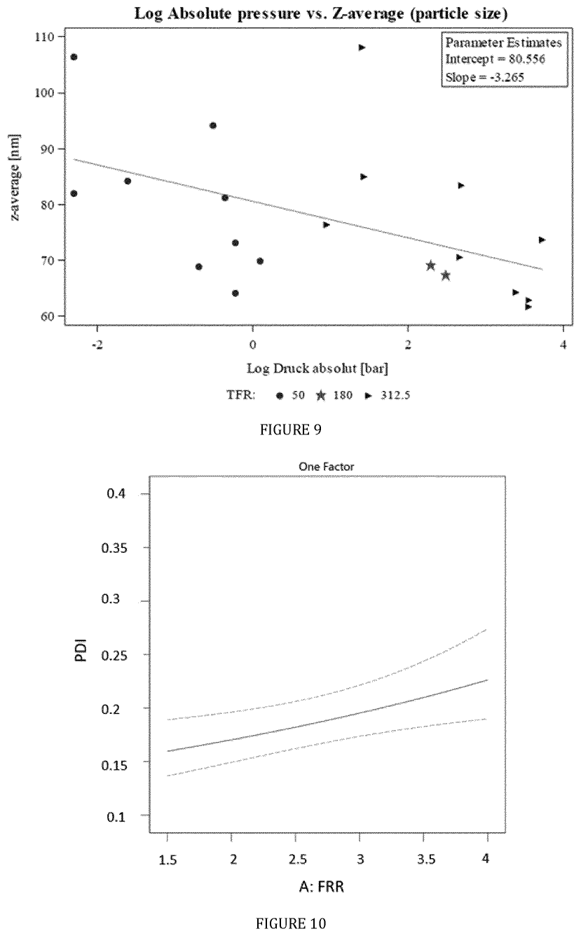

The DoE revealed that three of the investigated process parameters have an influence on particle size and PDI (see FIGS. 8, 9 and 10). The statistical power associated with the models used in the statistical analysis (Design Expert v.12 is the software) is above 80% and a P-value<0.05 was considered as statistically significant. Statistical analyses suggest that the following correlations exist between process parameters and particle attributes:
a. Nozzle size influences particle size: the larger the nozzle size of the reactor the larger the particle size.
b. Total pressure (TP) influences particle size: the higher the total pressure the smaller the particles.
c. FRR influences PDI: the smaller the FRR the higher the PDI.

Reactor & Total pressure (TP): over the working pressure range of the SYSTEM B equipment (0.1-41.7 bar), the impact of TP on the average particle size was moderate, with the average particle sizes being about 60% above the lowest average particle sizes. This shows that unloaded LNPs of the test formulation can be produced by jet impingement over a very broad range of pressure while retaining key characteristics such as uniform particle size (˜64-106 nm), stable PDI (0.12-0.25) and homogeneous morphology.

Flow rate ratio (FRR): A FRR range from 1:1.5 to 1:4 was tested. In principle, all FRRs within this range are suitable. At the same time, a correlation between FRR and PDI (P-value=0.0088; R2=0.32; FIG. 10) was observed. A FRR of 1:3 or 1:2 appears particularly useful for producing LNPs with a PDI<0.25, at least for the test formulation.

### Example 4—Preparation of mRNA-Loaded Lipid Nanoparticles

The pilot-scale equipment System B according to Table 1 above was used for the preparation of RNA-loaded lipid nanoparticles by the impingement jet method. The organic solvent stream comprised a model lipid composition with the lipids DLin-MC3/Cholesterol/DSPC/DMG-PEG2000 in ratios of 50/38.5/10/1.5 mol %, dissolved in EtOH as the organic solvent (with a total lipid concentration of 10 mg/ml). Poly(A) (polyadenylic acid) was used as a model RNA, and was provided in 50 mM citrate buffer (pH 6) at a concentration of 0.096 mg/mL, and used as the solution for the second (i.e. aqueous) stream.

The poly(A)-loaded LNPs were produced using an impingement jet reactor with nozzle opening diameters of 200 μm (first nozzle, organic liquid stream) and 400 μm (second nozzle, aqueous liquid stream). The suitability of different total flow rates (TFR) was investigated (40 ml/min, 120 ml/min, 280 ml/min), each at a flow rate ratio of 1:3 (organic stream to aqueous stream).

The particles were analyzed after dialysis using DLS (Stunner, Unchained labs, USA). Encapsulation efficiency was determined via RiboGreenassay.

Results: Lipid nanoparticles encapsulating Poly(A) within the expected size range, and with low PDI and high encapsulation efficiency (abbreviated as EE %) were generated at all total flow rates, indicating robustness of the process.

TABLE 3

| | | | | Concentration |
|---|---|---|---|---|
| TFR | Size | PDI | EE | encapsulated mRNA |
| [ml/min] | [nm] | [—] | [%] | (μg/ml) |
| 40 | 72.66 | 0.08 | 99.2 | 24.4 |
| 120 | 71.20 | 0.06 | 99.2 | 25.2 |
| 280 | 76.38 | 0.09 | 98.4 | 28.0 |

DLS measurements were also performed on samples prior to dialysis to determine stability and impact on ethanol concentration before dialysis. Samples which were diluted to 10% ethanol immediately after production as well as samples without additional dilution (25% ethanol) were analyzed approximately 3 h after sample production and storage under refrigerated temperatures.

TABLE 4

| TFR | Size [nm] | PDI [—] | Size [nm] | PDI [—] |
|---|---|---|---|---|
| [ml/min] | 10% EtOH | 10% EtOH | 25% EtOH | 25% EtOH |
| 40 | 58.68 | 0.13 | 62.92 | 0.10 |
| 120 | 60.15 | 0.19 | 63.30 | 0.14 |
| 280 | 66.75 | 0.20 | 68.69 | 0.13 |

As shown in Table 4, minimal change in particle size of the samples stored for 3 h without additional dilution was observed. The PDI was smaller or comparable for the samples without ethanol dilution; generally suggesting a good stability of the LNPs prior to dialysis, also with no dilution.

### Example 5—mRNA Integrity Assessment

Method: An aqueous stream of firefly luciferase (FLuc) mRNA (APExBIO) was circulated through one side of the System B equipment through the 400 μm pinhole of a 200/400 μm impingement jet reactor (the 200 μm nozzle was blocked with a plug) at a high pump speed of 5500 rpm and at a pressure of 6-7 bar to create significant shear forces. Samples were withdrawn at intervals (2 sec-4 min) and analyzed using an automated gel electrophoresis (Bioanalyzer). Samples were withdrawn after the following timepoints and analyzed in separate lanes: t=0 (before start), (after prime), 2 sec, 5 sec, 10 sec, 20 sec, 30 sec, 1 min, 2 min, 3 min, 4 min.

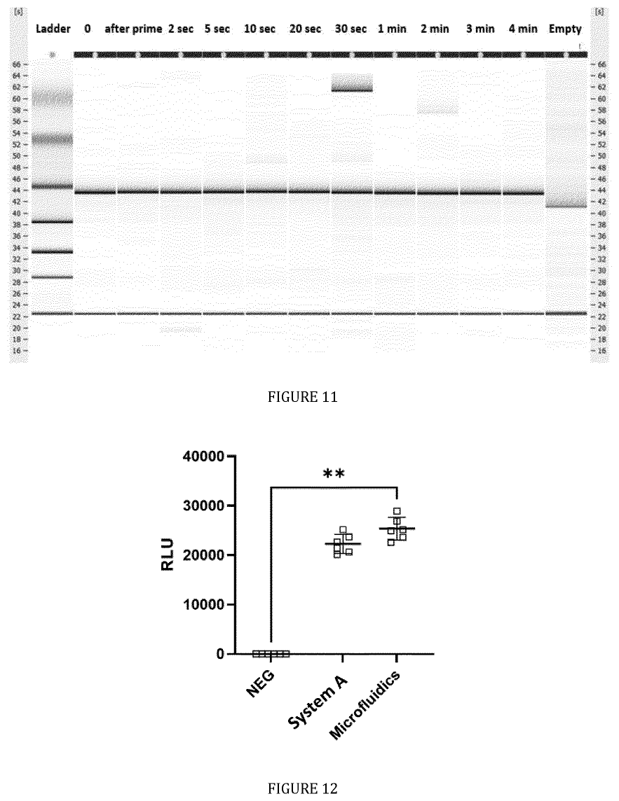

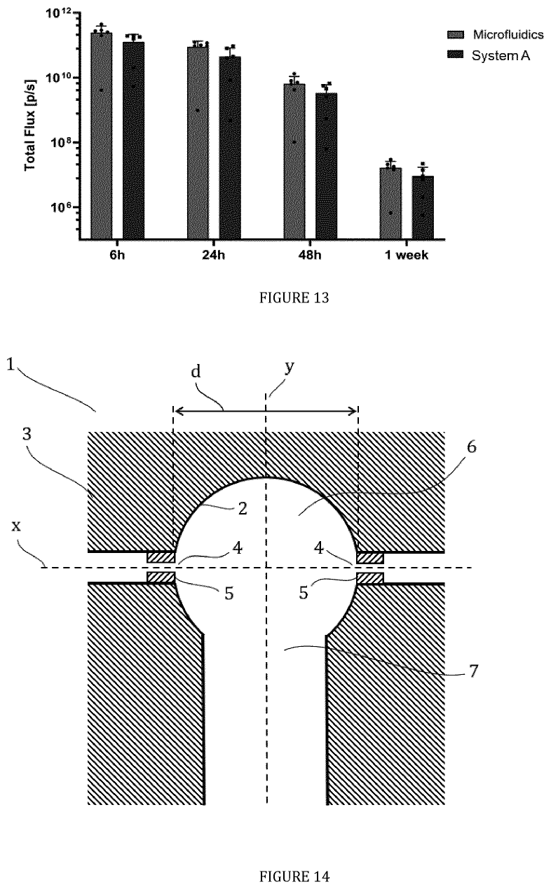

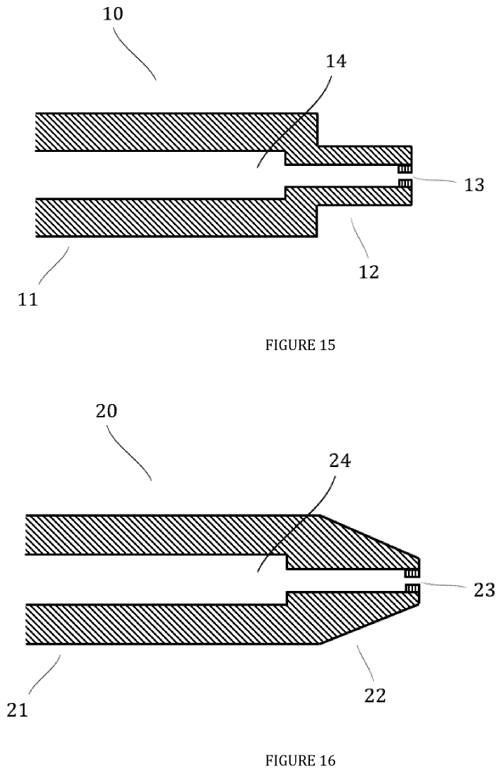

Results: Circulating the mRNA through the equipment system did not lead to detectable mRNA degradation irrespective of the circulation time. One complete passage through the System B equipment takes approx. 2 seconds. Thus, a circulation time of 4 minutes corresponds to approx. 120 passages through the nozzle, at high pump speed and pressure. Even after 4 mins (see FIG. 11) the mRNA had retained its integrity, showing that the pressure and shear force conditions encountered when manufacturing mRNA-loaded lipid nanoparticles according to the method disclosed herein are not harmful for the mRNA.

### Example 6—Preparation of Lipid Nanoparticles Comprising FLuc mRNA

Lipid nanoparticles comprising firefly luciferase (FLuc) mRNA were prepared with the jet impingement method using bench scale equipment System A, with nozzle opening diameters of 100 μm (first nozzle, organic liquid stream) and 200 μm (second nozzle, aqueous liquid stream). As a comparison, particles were also produced using a laminar flow based microfluidic mixing equipment (Ignite™, Precision Nanosystems). Transfection performance of the lipid nanoparticles produced using these systems was also tested.

Method: The active ingredient-free Onpattro® lipid formulation DLin-MC3/Cholesterol/DSPC/DMG-PEG2000-50/38.5/10/1.5 mol % in EtOH as organic solvent (with a total lipid concentration of 10 mg/ml) was used as the organic (i.e. first) stream. FLuc mRNA (Trilink or APExBIO) at a concentration of 0.09 mg/ml in 50 mM citrate buffer (pH 4) was used as the aqueous (i.e. second) stream. A flow rate ratio (FRR) of 1:3 (organic stream to aqueous stream) was used, at total flow rate of 30 ml/min with the jet impingement system, and a total flow rate of 10 ml/min with the microfluidic equipment. The particle size (z-average) and polydispersity index (PDI) of the particles were determined by dynamic light scattering (DLS) using a Stunner instrument (Unchained Labs). Cryo-transmission electron microscope (TEM) samples were inspected on a Tecnai F20 TEM. Encapsulation efficiency was as determined and quantified using a fluorogenic Quant-iT™ RiboGreen™ RNA assay kit (Thermo Fisher Scientific).

For the in vitro transfection assay, HepG2 cells were incubated in a 24-well plate format with various doses of FLuc mRNA-loaded lipid nano-particles. An in vivo transfection assay was also conducted: B6 albino mice (n=6 per group) received a single IV (tail vain) injection of 60 μg mRNA-loaded lipid nanoparticles (2.4 mg/kg). Bioluminescence imaging on an IVIS Spectrum imaging system (PerkinElmer) was performed 6 h, 24 h & 48 h after administration.

Results. The FLuc-mRNA loaded lipid nanoparticles prepared according to the impingement method were found to have useful particle sizes, PDI, and encapsulation efficiency. The respective values were similar to those measured for the nanoparticles obtained using the comparator laminar flow microfluidic system (see Table below).

| | | | Encapsulation |
|---|---|---|---|
| | Avg. | Avg. | Efficiency |
| Equipment | Z-Average [nm] | PDI [—] | EE [%] |
| System A | 76.8 | 0.11 | 97.5 |
| Microfluidic System | 75.6 | 0.11 | 97.7 |

Cryo-TEM (Tecnai F20 TEM) analysis of a sample of the lipid nanoparticles prepared using System A showed spherically-shaped particles. The high encapsulation efficiency of >97% of the Fluc mRNA translated into high transfection rates in the cell assay (see FIG. 12, showing in vitro transfection assay using 10 μg mRNA according to EE % assay per transfection. All data n=6, non-parametric, Kruskal-Wallis post AVG+/−SD, **p<0.01) as well as the in vivo assay (see FIG. 13, showing in vivo gene delivery quantification for total body over time), where FLuc expression was observed to peak at 6 h post-dosing and to decline thereafter in both tested groups. In vivo FLuc bioluminescence imaging suggested that there was systemic delivery of the of the Fluc mRNA by the lipid nanoparticles produced by both systems.

### Example 7—Encapsulation of pDNA

The use of the method according to the present disclosure to prepare nanoparticle dispersions comprising or encapsulating larger nucleic acid products such as plasmid DNA (pDNA) was also tested.

Method: Lipid nanoparticles loaded with pDNA were prepared using System A equipment with a impingement jet reactor fitted with nozzles having openings of 100 μm (first nozzle, for organic stream) and 200 μm (second nozzle, aqueous stream) in diameter. 50 mM Citrate buffer (pH 4) further comprising a stock solution of firefly luciferase pDNA (9790 bp, 0.9 mg/mL) was provided as the aqueous stream, and two lipid formulations were provided, dissolved in ethanol, and each was used individually as the organic stream: a formulation comprising the lipids DLin-KC2-DMA/cholesterol/DOPE/DMG-PEG2000 at a ratio of 50/38.5/10/1.5 mol % in ethanol (‘Formulation A’; total lipid concentration of 10 mM), and a lipid composition comprising DLin-MC3-DMA/cholesterol/DSPC/DMG-PEG2000 at a ratio of 50/38.5/10/1.5 mol % in ethanol (‘Formulation B’, total lipid concentration of 10 mM).

The same lipid composition as Formulation A but at a total lipid concentration of 17.2 mM in ethanol (‘Formulation C’) was also used for the encapsulation of firefly luciferase mRNA (Trilink; mRNA (5-methoxyuridine; 1929 nucleotides).

Encapsulation of the pDNA or mRNA was performed at a total flow rate (TFR) of 30 ml/min and at a flow rate ratio (FRR) of 1:3 (organic stream to aqueous stream). Samples were diluted to 10% ethanol with 50 mM citrate buffer (pH 4) after collection, dialyzed, and filtered. Particle size and PDI was determined by DLS (Stunner, unchained labs). Encapsulation efficiency (EE %) was determined using dsDNA Assay Kit for DNA and RNA Assay Kit of mRNA (Invitrogen), following manufacturer's protocol.

### Results

Lipid nanoparticles encapsulating pDNA were obtained from the impingement method according to the present disclosure, with a surprisingly high encapsulation efficiency. This was observed with both lipid compositions. The lipid particles comprising plasmid DNA, due to the larger size of these molecules had larger particle sizes than the mRNA.

| Sample | TFR | FRR | Size [nm] | PDI | EE [%] |
|---|---|---|---|---|---|
| mRNA | 30 | 3:1 | 79.28 | 0.07 | 98.1 |
| Formulation C | | | | | |
| pDNA | 30 | 3:1 | 119.38 | 0.10 | 95.0 |
| Formulation A | | | | | |
| pDNA/ | 30 | 3:1 | 108.21 | 0.18 | 97.2 |
| Formulation B | | | | | |

Further analysis of the nanoparticles by CryoTem analysis was performed and showed that in general, spherical particles were obtained. Further in vivo assays are performed to assess transfection efficiency of the produced lipid nanoparticle suspensions in which in vivo potency of the tested formulations is expected to be observed.

### Example 8—Preparation of Lipid Nanoparticles with a Jet Impingement Reactor

Model lipid nanoparticles, in analogy or in similar experiments (unloaded and/or loaded with test nucleic acid molecules) to those described herein above are prepared using an impingement jet reactor comprising a reaction chamber (6) defined by an interior surface (2) of a reaction chamber wall (3), the reaction chamber (6) having a substantially spheroidal overall shape, said chamber (6) comprising (a) a first and a second fluid inlet (4), wherein the first and the second fluid inlet (4) are arranged at opposite positions on a first central axis (x) of the reaction chamber (6) such as to point at one another, and wherein each of the first and the second fluid inlet (4) comprises a nozzle (5, 13, 23); and (b) a fluid outlet (7) arranged at a third position, said third position being located on a second central axis (y) of said chamber (6), the second central axis (y) being perpendicular to the first central axis (x); wherein the distance (d) between the nozzle (5, 13, 23) of the first fluid inlet (4) and the nozzle (5, 13, 23) of the second fluid inlet (4) is the same or smaller than the diameter of the reaction chamber (6) along the first central axis (x), and as described according to the embodiments of the present disclosure. It is expected that dispersions of lipid nanoparticles with low polydispersity and consistent particle sizes will be obtained in a reproducible manner and over a wide range of total flow rates, comparable to the particles obtained with other jet impingement equipment as described above.

1. A method for producing a dispersion of nanoparticles comprising at least one amphiphilic lipid, wherein the method comprises the steps of:
a) providing a first stream comprising an organic solvent and the amphiphilic lipid;
b) providing a second stream comprising an aqueous solvent;
c) pumping the first stream under a raised pressure through a first nozzle and pumping the second streams under a raised pressure through a second nozzle into a reaction chamber; wherein the first nozzle is located at an angle of about 180° from the second nozzle;
d) colliding the first stream and the second streams frontally in a reaction chamber; and
wherein the flow rate ratio between the first stream and the second stream is in a range from 1:1.5 to 1:4.5.

2. The method according to claim 1, wherein the flow rate ratio between the first stream and the second stream is in a range from 1:1.5 to 1:4, or from 1:2 to 1:4.5, or from 1:2 to 1:4, or wherein the flow rate ratio between the first stream and the second stream is about 1:1.5, 1:2, 1:2.5, 1:3, 1:3.5, 1:4 or 1:4.5.

3. The method according to claim 1 or 2, wherein the total flow rate is in the range of from 1 ml/min to 1,000 ml/min, or from 5 ml/min to 800 ml/min.

4. The method according to any one of the claims 1 to 3, wherein the total flow rate is in the range of from 100 ml/min to 500 ml/min and wherein the flow rate ratio between the first stream and the second streams is in a range from 1:1.2 to 1:2.5, or in a range from 1:2 to 1:4.

5. The method according to any one of claims 1 to 4, wherein the first nozzle comprises a first opening and the second nozzle comprises a second opening, wherein the first opening and the second opening have a diameter in the range of 40 μm to 800 μm.

6. The method according to claim 5, wherein the diameter of the first or second opening is at least about 50% larger than the diameter of the other opening.

7. The method according to any one of claims 1 to 6, wherein the pressure of the first stream is lower than or equal to the pressure of the second stream.

8. The method according to any one of claims 1 to 7, wherein the nanoparticles are selected from the group consisting of unilamellar liposomes, multilamellar liposomes, single bilayer liposomes, double bilayer liposomes, multivesicular liposomes, lipid nanoparticles, lipoplexes, and lipopolyplexes.

9. The method according to any one of claims 1 to 8, wherein the amphiphilic lipid is selected from the group of fatty acids, glycerolipids, monoglycerides, diglycerides, glycerophospholipids, phospholipids, phosphatidylcholine, soybean phosphatidylcholine (SPC), phosphatidylethanolamine, phosphatidylserine, sphingolipids, sterols, cholesterol, prenols, carotenoids, retinol, retinal, retinoic acid, beta-carotene, tocopherol, saccharolipids, LIPOID S100, PEGylated lipids, and 1,2-dimyristoyl-rac-glycero-3-methoxypolyethylene glycol (DMG-PEG).

10. The method according to any one of claims 1 to 9, wherein the first stream further comprises at least one further amphiphilic lipid.

11. The method according to claim 10, wherein the first stream comprises an amphiphilic lipid or a combination of lipids selected from the group of fatty acids, glycerolipids, monoglycerides, diglycerides, glycerophospholipids, phospholipids, phosphatidylcholine, soybean phosphatidylcholine (SPC), phosphatidylethanolamine, phosphatidylserine, sphingolipids, sterols, cholesterol, prenols, carotenoids, retinol, retinal, retinoic acid, beta-carotene, tocopherol, saccharolipids, LIPOID S100, PEGylated lipids, 1,2-dimyristoyl-rac-glycero-3-methoxypolyethylene glycol (DMG-PEG), and any combination thereof.

12. The method according to claim 11, wherein the first stream comprises a PEGylated lipid, cholesterol, and a cationic lipid, and a phospholipid.

13. The method according to any one of claims 1 to 13, wherein the first stream comprises an organic solvent selected from an alcohol, a ketone, a halogenated solvent, an amide, or an ether; optionally wherein the solvent is an alcohol, preferably ethanol.

14. The method according to any one of claims 1 to 13, wherein the first stream comprises a PEGylated lipid at a concentration in the range of 0.1 mol % to 2 mol %.

15. The method according to any one of claims 1 to 14, wherein the first or the second stream further comprises an active pharmaceutical ingredient.

16. The method according to claim 15, wherein the active pharmaceutical ingredient is selected from the group of small molecules, peptides, and nucleic acids.

17. The method according to claim 16, wherein the second stream further comprises a nucleic acid, preferably selected from the group consisting of DNA and RNA, optionally wherein the RNA is selected from the group consisting of mRNA, miRNA, pre-miRNA, saRNA, shRNA, siRNA, ribozyme, and antisense RNA; and further optionally from antigen-encoding DNA and mRNA.

18. The method according to any one of claims 1 to 17, further comprising a step of
purifying the nanoparticle dispersion by filtration;
subjecting the nanoparticle dispersion to lyophilization; or
removing the organic solvent from the nanoparticle dispersion by cross-flow filtration.

19. The method according to any one of claims 1 to 18 wherein the average particle size of the nanoparticles is from 20 nm to 100 nm.

20. The method according to any one of claims 1 to 19, comprising a step of providing a jet impingement reactor that comprises the reaction chamber, the first nozzle and the second nozzle.

21. The method according to claim 20, wherein each of the first and the second nozzle is connected with a feed line, and wherein each feed line is optionally associated with a pump arranged for providing the first and the second stream under raised pressure to the respective nozzle.

22. The method according to any one of the preceding claims, wherein the pressure of each stream is independently selected from a value in the range of 0.1 to 120 bar, and optionally from a range of 1 to 40 bar.

23. The method according to any one of the preceding claims, wherein the reaction chamber (6) is defined by an interior surface (2) of a reaction chamber wall (3), the reaction chamber (6) having a substantially spheroidal overall shape, said chamber (6) comprising:
(a) a first and a second fluid inlet (4), wherein the first and the second fluid inlet (4) are arranged at opposite positions on a first central axis (x) of the reaction chamber (6) such as to point at one another, and wherein the first fluid inlet comprises the first nozzle and the second fluid inlet (4) comprises the second nozzle (5, 13, 23); and
(b) a fluid outlet (7) arranged at a third position, said third position being located on a second central axis (y) of said chamber (6), the second central axis (y) being perpendicular to the first central axis (x);
wherein the distance (d) between the first nozzle (5, 13, 23) and the second nozzle (5, 13, 23) is the same or smaller than the diameter of the reaction chamber (6) along the first central axis (x).

24. The method according to claim 24, wherein the first nozzle (5, 13, 23) and the second nozzle (5, 13, 23) each have a downstream end (12, 22), and wherein the downstream end (12, 22) of each nozzle (5, 13, 23) substantially aligns with the interior surface (2) of the chamber wall (3), and/or wherein the nozzles (5, 13, 23) are arranged such as to direct the first and the second stream along the first central axis (x) towards the centre of the chamber (6) and to allow the first stream and the second stream to collide at an angle of about 180°.

25. The method according to claim 23 or 24, wherein
(i) the reaction chamber (6) has an overall shape of a spherical cap having a height, a basis, and a radius along the first central axis (x), wherein the height is larger than said radius, the height preferably being from 110% to 170% of said radius, and wherein the basis is defined by the fluid outlet (7), and/or
(ii) essentially all of the interior surface (2) of the reaction chamber wall (3) is substantially spherical, optionally with the exception of portions of the interior surface (2) that are part of the first and/or second fluid inlet (4) or of the fluid outlet (7); and/or
(iii) the reaction chamber (6) is free of other inlet or outlet openings.

26. The method according to any one of claims 23 to 25, wherein the reaction chamber (6) has a volume of not more than 0.25 mL and the distance (d) between the nozzle (5, 13, 23) of the first fluid inlet (4) and the nozzle (5, 13, 23) of the second fluid inlet (4) is not more than 5 mm.

27. The method according to any one of claims 23 to 26, wherein each of the first and the second fluid inlet (4) is provided by a fluid inlet connector (10, 20) having an upstream end (11, 21), a downstream end (12, 22) holding the nozzle (5, 13, 23) of the first or second fluid inlet (4), and a fluid conduit (14, 24) for conducting a fluid from the upstream end to the downstream end, and wherein the downstream end of each fluid inlet connector (10, 20) is reversibly insertable into the chamber wall (3) such as to provide the first and the second fluid inlet (4); wherein the fluid inlet connector (10, 20) that provides the first and/or the second fluid inlet (4) is optionally affixed to the chamber wall (3) by means of a single ferrule fitting or a double ferrule fitting.

28. The method according to claim 27, wherein the fluid inlet connector (10, 20) has
an upstream segment comprising the upstream end (11, 21) of the fluid inlet connector (10, 20) and an upstream portion of the fluid conduit (14, 24); and
a downstream segment comprising the downstream end (12, 22) of the fluid inlet connector with the nozzle (5, 13, 23) and a downstream portion of the fluid conduit (14, 24), wherein the diameter of the upstream portion of the fluid conduit (14, 24) is larger than the diameter of the downstream portion of the fluid conduit (14, 24).

29. The method according to any one of claims 23 to 28 wherein the first nozzle (5, 13, 23) and/or the second nozzle (5, 13, 23) is a plain-orifice nozzle (5, 13, 23) which is optionally made of sapphire, ruby, diamond, ceramic, or steel.

30. The method according to any one of claims 23 to 29 wherein the first nozzle (5, 13, 23) has a first orifice diameter and the second nozzle (5, 13, 23) has a second orifice diameter, wherein the first orifice diameter and/or the second orifice diameter is in the range of 20 μm to 500 μm, and wherein second orifice diameter is optionally larger than the first orifice diameter, the ratio of the second orifice diameter to the first orifice diameter optionally being from 1.2 to 5.

31. The method according to any one of claims 23 to 30, wherein the ratio of the diameter of the reaction chamber (6) along the first central axis (x) to the second orifice diameter is in the range from 6 to 60.

32. The method according to any one of claims 23 to 31, wherein the ratio of the diameter of the reaction chamber (6) along the first central axis (x) to the diameter of the fluid outlet (7) is in the range of about 1.2 to 3.

33. The method according to any one of claims 23 to 32 wherein the interior surface (2) of the reaction chamber wall (3) exhibits a surface roughness of not more than 0.8 Ra, wherein Ra is determined according to ISO 4287:1997.

34. The method according to any one of claims 23 to 33, wherein each of the first and the second stream is forced through the respective nozzle (5, 13, 23) at a pressure in the range of 0.1 to 120 bar, and optionally at a pressure in the range of 1 to 40 bar; and wherein optionally each of the first and the second fluid stream is directed into the reaction chamber (6) at a flow rate in the range of about 1 to 1000 mL/min.

35. The method according to any one of claims 23 to 34 wherein
the orifice of the second nozzle (5, 13, 23) is larger than the orifice of the first nozzle (5, 13, 23); and/or
the flow rate of the second stream is larger than the flow rate of the first stream;
and wherein the pressure of the first stream and of the second stream is adapted such as to cause the first stream and the second stream to have substantially the same kinetic energy when entering the reaction chamber, wherein the kinetic energy is optionally calculated according to the formula Eₖ=½*m*v².
